SEQUENCE LISTING (6 sequences)

SEQ ID NO: 1 - DNA, 1500 bp, Rattus norvegicus
  promoter (2)..(710): Contain the ANF promoter region to construct pANF-638Luc
gaattctttagagcctgtatcatgttggcttcctggctgacttcatactctaaaaaaatataatagctctttcacctgactgctaacagggacatctagggtgggggtgggctgtctggggccagaggtccacccacgaggccaatgaatcaggtgtgaaggtaactccagtatgcgggctcccccgcagcctagctgtctcccagctgcctgtcattgcctctcctcccgcccttatttggagcccctgacagctgagatgcaagcagagggagctgggtgtgggccagccgtcaccctctgcttccctgcatgggtcccgttgccagggagaaggaatcctgaggcgagcgcccaggaagataaccaaggactcttttctgctcttctcacacctttgaagtgggggcctcttgaggcaaatcatcaagaatgtgactcttgcagctgagggtctgggggagggagggttactggagctgctcaaggcaaaggggctgtgacaagcttcgctggactgataactttaaaagggcatcttctgctggccgccgcaagtgacagaatggggagggttccagctctcctgcgttctcagggagctggggggctataaaaacgggagacgccgggcagctgggagacagtgacggacaaaggctgagagagaaaccagagagtgagccgagacagcaaacatcagatcgtgccccgacccacgccagcatgggctccttctccatcaccaagggcttcttcctcttcctggccttttggctcccaggccatattggagcaaatcccgtatacagtgcggtgtccaacacagatctgatggatttcaaggtagggccaggaagtggggcatggactgggaccagggtctccttggtactgggtccattcctgagacatccccctttctctgcatttattttcccctgataaagaacctgctagaccacctggaggagaagatgccggtagaagatgaggtcatgcctccgcaggccctgagcgagcagaccgatgaagcgggggcggcacttagctccctctctgaggtgcctccctggactggggaagtcaacccgtctcagagagatggaggtgctctcgggcgcggcccctgggacccctccgatagatctgccctcttgaaaagcaaactgagggctctgctcgctggccctcggagcctgcgaaggtcaagctgcttcgggggtaggattgacaggattggagcccagagcggactaggctgcaacagcttccgggtaagaggcgctgcgggtgaaacgggatagaggccaggtggggtcttgttagggctccgaccttgccaaggactagtgccagtctgcatcttcggcagtacagagtccagtgcgtgagtcttatgttctctgagagttctgccccaccctgatgggtgtcccttgagtttcaagagaatgacagcagctgctgcaggatctgagccacgagcactgggaaatt

SEQ ID NO: 2 - DNA, 86 bp, Rattus
  promoter (1)..(86): Fragment from the alpha MHC promoter
gtcccagcagatgactccaaatttaggcagcaggcacgtggaatgagctataaaggggctggagcgctgagagctgtcagaccgag

SEQ ID NO: 3 - DNA, 35 bp, GATA4  Enhancer
caaagggccgatgggcagatagaggagagacagga

SEQ ID NO: 4 - DNA, 1588 bp, Rattus
gaattctcttactatcaaagggaaactgagtcatgcacctgcaaaatgaatgccctccctggacatcatgactttgtccctggggagccagcactgtggaactccaggtctgagagtaggaggcacccctcagcctgaagctgtgcagatagctagggtgtaaaagagggaaggggggaggctggaatgggagcttgtgtgttcggagacaggggacaaatattaggcccgtaagagaaggtgacccttacccagtgtgttcaactcagcctttcagattaaaaataactaaggtaagggccatgtgggtaggggaggtggtgtgagacggtcctgtctctcctctatctgcccatcggccctttggggaggaggaaatgtgcccaaggactaaaaaaggcctggagccagaggggctagggctaagcagacctttcatgggcaaacctcagggctgctgtcctcctgtcacctccagagccaagggatcaaaggaggaggagccagacaggagggatgggagggagggtcccagcagatgactccaaatttaggcagcaggcacgcggaatgagctataaaggggctggagcgctgagagctgtcagaccgagatttctccatcccaagtaagaaggagtttagcgtgggggctctccaaccgcaccagacctgtcccacctagagggaaagtgtcttccctggaagtgggctcctcccaccggcctgggaagattcctcggtgggcaggatgttctactggatgccccttcccttccactgcctcctccctcccttgtcttgattaatcttggctcttagtgttcagaaagatttgcccggtgctgtctactccatctgtctctactctctctgccttgccttcttgtgtgttctccttttccacgtgtttctcactccactgcctcccccccccccttcatttttatccttcctttctttctgtgtcagaatgctgggaatcaaacccagggcttcatacacgtcaagtaagcaatctcccagtgagtcaaagctttaatcctctgggtgctgtcttaccgagcctcactccctgtcttgtcctgttccgtcctagtcaggatctctggtccgtctctcagcttctgctactcctctccctgcctgctcttctctccgtccagctgcacctctgtggcgctcattccagccgtggtccaaattctctgtgaaaagattaaccgggtgagaatgcccccagtttcccctgtagacagcagatcatgattttccccagaagccagacttccagcgcccgccctctgcccagcaacttgacactcttagcaaacttcagccacccttcccccacatagaccaagtcttgcagagagccttccttcagatgacttcgagttcttgcaaaggaaggagaactctttgtggcggggaagcaggcactttacacggagtctgacgggaggtcataggctatggcatagcagaggcagggaggtggtggaattggacttcgcgcagaagctaagcacacaccaggaatgacatatccctcctatctcccccataagagtttaagagtgacagg

SEQ ID NO: 5 - DNA, 1679 bp, Mouse
gaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtgacaga

SEQ ID NO: 6 - DNA, 118 bp, Homo sapiens
cgaaggggaccaaataaggcaaggtggcagaccgggccccccacccctgcccccggctgctccaactgaccctgtccatcagcgttctataaagcggccctcctggagccagccaccc

(19) United States
(12) Patent Application Publication
(10) Pub. No.: US 2003/0204206 A1
(43) Pub. Date: Oct. 30, 2003

(54) Electrically responsive promoter system

(75) Inventors: Rodolfo A. Padua, Richfield, MN (US); Carl A. Schu, Plymouth, MN (US); Matthew D. Bonner, Plymouth, MN (US); Maura G. Donovan, St. Paul, MN (US); Orhan Soykan, Houghton, MI (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/027,655

(22) Filed: Dec. 20, 2001

Related U.S. Application Data
(60) Provisional application No. 60/257,460, filed on Dec. 21, 2000.
(60) Provisional application No. 60/313,926, filed on Aug. 20, 2001.

(51) Int. Cl. A61N 1/36

(52) U.S. Cl. 607/2

(57) ABSTRACT

The present invention provides methods and systems for regulating delivery of therapeutic proteins and nucleic acids. Specifically, this involves using a genetically engineered electrically responsive promoter operably linked to a therapeutic gene sequence, wherein expression of said sequence is controlled by an electrical pulse generator

### FIELD OF THE INVENTION

The present invention provides novel systems, components, and methods that control and regulate production of therapeutic products. More specifically, the present invention provides electrically responsive promoters operably coupled to an electrical pulse generator for the production of therapeutically useful products, and devices related thereto.

### BACKGROUND OF THE INVENTION

Over time a number of recombinant systems have been developed to produce therapeutic proteins exogenously. The recombinantly produced proteins were isolated and purified and then systemically delivered to a patient. This approach has resulted in delivery of some important therapeutic proteins (e.g., erythropoietin, interferon, insulin) but has failed to be a generally applicable approach, most notably because of problems associated with protein stability. Others have addressed this problem by focusing on fluid delivery systems (catheters, syringes) for local protein or gene delivery. Others have sought to use cell transplantation to provide in vivo delivery of therapeutically useful products. Alternatively, others have developed viral based gene delivery systems to directly produce the desired therapeutic gene or protein in vivo.

To this end, recombinant vectors and viruses have been developed to effectively introduce and express genes in many cell types. The requirements for successful gene therapy include stable and safe vectors, elements that promote long-term expression, and the ability to regulate the expression of the gene of interest for the purpose of controlling the dose and duration of the targeted therapeutic product. Although extensive research continues in the areas of gene delivery, very little has been reported on methods to control and regulate gene expression in vivo.

Researchers have taken advantage of inherent DNA sequences found upstream of a gene, which regulate the expression of the gene under different physiological conditions. Several protocols have been published which have focused on pharmacologically-based control of gene expression. Generally the basis of these methods relies on the presence of a pharmacological agent to control the activation of the DNA promoter sequences. An example of this is the Tet-On/Tet-Off gene expression system, which is commercially available from Clontech. Presence or absence of tetracycline or doxycycline will activate the promoter responsible for turning on gene expression. Administration of the activating pharmacological agent is generally done systemically in an effort to deliver the agent affecting transcription to the site of the action. Although technically effective at inducing gene expression, the possibility exists that systemic administration of pharmacological agents in vivo can result in unwanted side effects or toxicity in surrounding tissues. Further, because pharmacological agents reside in the body over a period of time, often for days, regulation of the gene promoter sequence is not tightly coupled from the time the activating agent is given until it is eliminated from the body.

Until the present invention, controlled delivery of therapeutic gene products has not been regulated in a patient via an electrical device. In the present invention, an electrical pulse generator, e.g., a pacemaker, is used to closely modulate the time, frequency, and delivery amount of a given therapeutic product and to closely define the locus of delivery. Under the present invention, tissues containing genetically engineered cells which have received electrically responsive promoter elements direct the expression of a therapeutic product upon receiving electrical stimulation. The present invention describes a novel system to utilize an electrical stimulus (provided by an electrical pulse generator) as a means to control the expression of electrically responsive promoters (ERPs) that have been transplanted or incorporated into the tissue of a mammal. The target gene of interest is operably linked to an electrically responsive promoter sequence to provide controlled expression by the ability to closely regulate the electrical stimulus. The ERP gene constructs can be delivered by standard gene transfection methods to cells grown in culture and then implanted into the patient, or delivered directly to tissues or cells in vivo through the use of an appropriate gene delivery vector (viral or non-viral). Implantable electrodes operably coupled to the pulse generator can then be used to electrically stimulate at a defined locus the electrically responsive promoters in transfected or transplanted cells, which consequently results in the controlled expression of operably linked DNA sequences.

### SUMMARY OF THE INVENTION

The present invention has certain objects that address problems existing in the prior art with respect to controlled and local delivery of therapeutically important products. Various embodiments of the present invention provide solutions and advantages to one or more of the problems existing in the prior art with respect to delivery of therapeutic products. To each of the embodiments the present invention provides one or more particular features that is taught or further illustrated herein.

The present invention provides novel electrically responsive systems for production of therapeutically useful gene or protein products. In another aspect it provides a new delivery means for existing products as well as for developing products.

The invention also provides electrically responsive promoter elements linked to a pulse generator in a patient in need thereof. In one aspect, the invention includes a method for reducing or repairing tissue injury by providing a means for delivery of therapeutic proteins. The delivery system is effective in repairing tissue injury, such as ischemic injury. The method may be applied to damaged cardiac tissue, kidney tissue, brain tissue, or endothelial tissue by providing a therapeutic gene operably linked to an electrically responsive promoter.

In one embodiment the present invention provides methods for introducing into at least one cell a chimeric gene containing an electrically responsive element operably linked to a promoter to control transcription of the therapeutic gene in a cell, wherein the electrical response element is capable of modulating gene expression of the therapeutic gene upon exposure to electrical stimulation to produce a therapeutic product.

The present invention also provides a delivery system whereby the therapeutic agent is delivered at the locus of the target tissue by directed placement of the electrical stimulus. The present invention also provides directed delivery of therapeutic products by directed placement of the electrically responsive promoter containing cells.

In one aspect the electrically responsive system provides an electrical pulse generator operably coupled to genetically engineered cells containing electrically responsive promoter elements operably linked to a gene. In one feature, the pulse generator is capable of providing a course of subthreshold stimulation to the targeted tissue.

In one embodiment, the present invention provides a system that is capable of stimulating cells for controlled expression of therapeutically useful gene and protein sequences. In a related aspect, the invention includes a chimeric gene, containing an electrical response element which is heterologous to the therapeutic gene. Alternatively, the electrical response element is heterologous to the promoter. In either case, the electrically responsive element is operably linked to the promoter to effectively modulate expression of the therapeutic gene. The method may be used with a variety of cell types and corresponding promoters. In one preferred embodiment the cells are muscle cells, and more preferably, heart or skeletal muscle cells. Another aspect of the present invention includes the above-described chimeric gene carried in an expression vector. The expression vector may be a plasmid, adenovirus vector, retrovirus vector, or the like.

In other embodiments, the present invention provides a novel testing device and method for testing and finding electrically responsive promoters.

These and other objects and features of the invention will become more fully apparent when the following detailed description is read in conjunction with the accompanying drawings.

### BRIEF DESCRIPTION OF THE DRAWINGS

The following drawings depict certain embodiments of the invention. They are illustrative only and do not limit the invention as otherwise disclosed herein.

FIG. 1: Electrical Stimulation of Electrically Responsive Promoters in Transfected Tissues for Production of Therapeutic Products.

FIG. 1 is an overview of one mode of operation of electrically responsive promoters to produce a therapeutic product. Schematically shown are transfected or transplanted heart cells containing electrically responsive promoters that, upon electrical stimulation, produce a therapeutic product.

FIG. 2. Electrical Stimulation of Electrically Responsive Promoter Cells Carried on a Stent.

FIG. 2 is an illustration of an implantable system according to the present invention that includes the use of a radio frequency (RF) signal to communicate and generate an electrical current in a coiled stent. Inset FIG. 2A is a diagrammatic representation of a circuit in a coiled stent for electrically stimulating electrically responsive promoter cells in association with the stent.

FIG. 3. Delivery of Electrically Responsive Promoter Cells on Scaffolding.

FIG. 3 shows two alternate constructions for delivering an applied electric field to engineered cells grown on a scaffolding: (A) a conductive matrix having parallel electrodes, and (B) a conductive stent matrix

FIG. 4: Electrically Responsive Vectors

FIG. 4 depicts a general vector construction of a therapeutic gene operably linked to an electrically responsive promoter. Also shown is the SV40 polyA tail and enhancer, and the ampicillin resistance gene for bacterial propagation.

FIG. 5: Expression Vectors pANF-65GL

FIG. 5 is a vector map of pANF-65GL. pANF-65GL was created from the parent vector, pGL2-promoter, by replacement of the viral promoter with ANF transcription start site (+1) and various lengths of 5′ flanking sequence. Shown are the multiple cloning sites upstream of ANF-65, into which electrical responsive elements (optionally with tissue specific and/or silencer elements) can be cloned;

the SV40 3′ untranslated region providing the polyadenylation signals 3′ to the luciferase coding region as well as 5′ to the promoter (An); and the ampicillin resistance gene for propagation in bacterial cells. In the particular constructs, the restriction endonuclease sites appearing in parentheses are no longer available due to modification created by the inserted DNA, e.g., Nhe 1 is unavailable for—134GL. The plasmid p638ANFluc was constructed from the parent vector pGL2 by replacement of the SV40 promoter with the ANF promoter from the start site (+1) to −638 of the 5′ flanking sequence

FIG. 6: Enhanced Expression from Electrically Responsive Promoters

FIG. 6 illustrates electrical stimulation enhanced the expression of luciferase in QBI-293A cells transfected with p638ANFluc. Cells were transfected with p638ANFluc as described herein. Twenty-four hours after transfection, cells were simulated for 24 hours under various conditions: (1) 10 Hz, 20 ms, 1 mA, 1.3 s polarity reversal; (2) 10 Hz, 10 ms, 4 mA, 6.0 s polarity reversal; (3) 10 Hz, 20 ms, 1 mA, 6.0 s polarity reversal; (4) 5 Hz, 5 ms, 2 mA, AC coupled; (5)10 Hz, 20 ms, 1 mA, AC coupled. After twenty-four hours of stimulation cells were harvested, and luciferase expression quantified.

FIG. 7: Time Course for Activation: QBI-293 Cells

FIG. 7 shows the time course of luciferase expression in p638ANFluc transfected QBI-293 cells after electrical stimulation. Cells were electrically stimulated at 10 Hz, 20 ms, 1 mA, 1.3 s polarity reversal. Electrical stimulation elicited a maximal 2.4 fold enhancement of luciferase expression after twenty-four hours, but enhanced expression was evident after 1 hour of stimulation.

FIG. 8: Time Course for Activation: C2C12 Cells

FIG. 8 shows the time course for activation of luciferase in C₂C₁₂ cells after electrical stimulation. Cells transfected with p638ANFluc were electrically stimulated (10 Hz, 20 ms, 1 mA, 1.3 sec polarity reversal) for various time points up to twenty-four hours. C₂C₁₂ cells showed near maximal enhancement of luciferase expression at 20 minutes of stimulation.

FIG. 9: In Vitro Apparatus for Electrical Stimulation

The test apparatus for testing promoter constructs is based on a modified 6-well polystyrene cell culture plate. FIG. 9 is a schematic representation of one of the wells as viewed from the side.

FIG. 10: Electrical Stimulation Sequence for In Vitro Testing

FIG. 10 shows a in vitro test apparatus for testing electrically responsive promoters (ERPs). This stimulation sequence consists of a train of 20 msec. pulses at a rate of 10 Hz. (100 msec. from one pulse to the next). The pulses are monophasic (not charge balanced), but the polarity of the pulses is reversed every 1.3 secs.

FIG. 11: Pulse Generator with Telemetry and Sensor Functionality

FIG. 11 shows schematically a pulse generator with telemetry and sensor functionality.

FIG. 12: Pulse Generator for Threshold Stimulation

FIG. 12 shows a block diagram of a circuit for pulse generator capable of delivering electrical stimulation to the target tissue cells.

FIG. 13: Simplified Schematic of the Output Circuit for Subthreshold Stimulation

FIG. 13 illustrates the schematic of the output circuitry of a subthreshold stimulation device for a pulse generator.

FIG. 14: Equivalent Circuit of the Subthreshold Stimulation During the Output Stage

FIG. 14 illustrates the schematic of the output circuitry of a subthreshold device for a pulse generator during the output stage.

FIG. 15: Subtheshold Stimulation Sequence

FIG. 15 illustrate a pacing scheme for providing a series of subthreshold stimulations.

### BRIEF DESCRIPTION OF THE SEQUENCES

SEQ ID NO:1 is the nucleotide sequence to the ANF promoter region of pANF-638Luc

SEQ ID NO:2 is the nucleotide sequence of the rat alpha MHC promoter fragment.

SEQ ID NO:3 is the nucleotide sequence of the sense strand of the GATA4 enhancer element.

SEQ ID NO:4 is the nucleotide sequence of the rat cardiac alpha-myosin heavy chain promoter region fragment.

SEQ ID NO:5 is the nucleotide sequence of mouse cardiac alpha-myoxin heavy chain promoter region.

SEQ ID NO:6 is the nucleotide sequence of the human cardiac actin promoter region.

### DETAILED DESCRIPTION OF THE INVENTION

Definitions

Molecular and Cell Biology

The term “genetically engineered cell(s)” refer to cells that have been had defined segments of nucleic acid purposefully introduced into the cell. The term “genetically engineered cell” is not meant to be limited by the means of introduction of the nucleic acid unless specifically so indicated..

“Host cell” refers to any eukaryotic or prokaryotic cell that is suitable for expressing a gene operably linked to an exogenously provided electrical response element. The electrical response element may be provided by transformation or transfection of either cells in culture or cells found in targeted tissues..

“Isolated nucleic acid compound” refers to any RNA or DNA sequence, however constructed or synthesized, which is removed from its natural location.

The term “mature protein” or “mature polypeptide” as used herein refers to the form(s) of the protein produced by expression in a mammalian cell. It is generally hypothesized that, once export of a growing protein chain across the rough endoplasmic reticulum has been initiated, proteins secreted by mammalian cells have a signal sequence which is cleaved from the complete polypeptide to produce a “mature” form of the protein. Often, cleavage of a secreted protein is not uniform and may result in more than one species of mature protein. The cleavage site of a secreted protein is determined by the primary amino acid sequence of the complete protein and generally cannot be predicted with complete accuracy. However, cleavage sites for a secreted protein may be determined experimentally by amino-terminal sequencing of the one or more species of mature proteins found within a purified preparation of the protein.

The term “operably linked”, as used herein, denotes a relationship between a regulatory region (typically a promoter element, but may include an enhancer element) and the coding region of a gene, whereby the transcription of the coding region is under the control of the regulatory region. As used herein, “operably linked” refers to a juxtaposition of transcriptional regulatory elements such that the transcriptional function of the linked components can be performed. Thus, an ERP promoter sequence “operably linked” to a coding sequence refers to a configuration wherein the promoter sequence promotes expression (or inhibits the expression if a negative regulatory element) of the gene sequence upon electrical stimulation.

“Operably coupled” refers to the transference of an electrical stimulus by an electrical pulse generator to a tissue. A pulse generator operably coupled with genetically engineered cells of the present invention refers to a configuration where an electrical stimulus is delivered to the tissue area containing genetically engineered cells to cause expression of an operably linked therapeutic product. Usually the stimulus is delivered from the pulse generator through leads to electrodes attached to the tissue.

An “electrically responsive promoter” or “ERP” is a promoter that contains a genetically engineered electrically responsive element that modulates transcription of the operably linked therapeutic product in a cell upon the delivery of an electrical stimulus. Modulated transcription may be positive or negative, and may change the relative transcriptional amount over time by an amount that is equal to or approximately 2, 4, 6, 10, 20, 50, 100, or 1000 fold or greater than unstimulated cells over 1, 2, 4, 8, 16, 24, 48, or 72 hours. In one embodiment the ERP promoter is an ANF promoter.

The term “promoter” refers to a nucleic acid sequence that directs transcription, for example, of DNA to RNA. As referred to herein the promoter includes the 5′ flanking sequences that promote transcription. A promoter may contain several regulatory sequences. A constitutive promoter generally operates at a constant level and is not regulatable. The ERP promoters of the present invention can be induced by electrical stimulation.

“Recombinant DNA cloning vector” as used herein refers to any autonomously replicating agent (including, but not limited to, plasmids and phages) comprising a DNA molecule into which one or more additional DNA segments can be or have been incorporated.

The term “recombinant DNA expression vector” or “expression vector” as used herein refers to any recombinant DNA cloning vector (such as a plasmid or phage), in which a promoter and other regulatory elements are present, thereby enabling transcription of the inserted DNA, which may encode a polypeptide.

The term “vector” as used herein refers to a nucleic acid compound used for introducing DNA into host cells. A vector comprises a nucleotide sequence which may encode one or more protein molecules. Plasmids, cosmids, viruses, and bacteriophages, in the natural state or which have undergone recombinant engineering, are examples of commonly used vectors. The term “vector” also applies to the use of viral vectors such as those further described herein.

The term “plasmid” refers to an extrachromosomal genetic element. The plasmids disclosed herein are commercially available, publicly available on an unrestricted basis, or can be constructed from readily available plasmids in accordance with published procedures.

An “element”, when used in the context of nucleic acid constructs, refers to a region of the construct or a nucleic acid fragment having a defined function. For example, an electrical response enhancer (ERE) element is a region of DNA that, when associated with a gene operably linked to a promoter, enhances the transcription of that gene under conditions where the cells of the tissue are provided an appropriate electrical stimulus.

Two nucleic acid elements are said to be “heterologous” if the elements are derived from two different genes, or alternatively, two different species. For example, an electrical response enhancer element from a human ANF gene is heterologous to a promoter from a human myosin gene. Similarly, an electrical response enhancer element from a human ANF gene, for example, is heterologous to a promoter from a mouse ANF gene.

“Chimeric gene,” also termed “chimeric DNA construct,” refers to a polynucleotide containing heterologous DNA sequences, such as promoter and enhancer elements operably linked to a therapeutic gene. For example, a construct containing a human alpha.-myosin heavy chain (alpha.-MHC) promoter fragment operably linked to a human bcl-2 gene and containing a human erythropoietin gene hypoxia response element comprises an exemplary chimeric gene.

“Target gene” refers to a gene whose transcription is operably linked to an electrically responsive promoter.

“Mammalian tissue” refers to the tissues of vertebrates that are well known generally to scientist. They include, but are not limited to cells of endodermal, ectodermal, or mesodermal origin, that make up such structures as heart muscle, blood vessels, nerve, bone, muscle, skin, pancreas, and the specialized cells that make up these tissues (See The Molecular Biology of The Cell, 3^{rd} Edition, 1994, Garland Publishing, pp. 1188-1189). For example, cells of the mesodermal origin that form contractile cells include skeletal muscle cells, heart muscle cells, and smooth muscle cells, as well precursor cells to the cells, such as pluripotent stem cells, mesodermal stem cells, myoblast, fibroblasts, and cardiomyocytes. Cells endodermal origin that help make up nervous tissue include, but are not limited to, autonomic neurons, cholinergic, andrenergic, and peptidergic neurons, gial cells (astrocytes, and oligodendrytes), as well as supporting cells of the peripheral nervous system, such as, schwann cells. Epithelial cells, include but are not limited to vascular endothelial cells of blood vessels and lymphatic systems, synovial cells, , and the like. A number of specialized cells of the pancreas, such as, acinar cells and cells of the liver, hepatocytes, and cells making up or surrounding bone tissue (chondrocytes, osteoblasts, osteoprogenitor cells, nucleous pulposus cells of the intervertebral disk), are also specifically included within the scope of the invention.

Medical and Other Terms

The terms “treating”, “treatment”, and “therapy” as used herein refer to curative therapy, prophylactic therapy, and preventive therapy. An example of “preventive therapy” is the prevention or lessening of a targeted disease or related condition thereto. Those in need of treatment include those already with the disease or condition as well as those prone to having the disease or condition to be prevented. The terms “treating”, “treatment”, and “therapy” as used herein also describe the management and care of a patient for the purpose of combating a disease, or related condition, and includes the administration of an ERP DNA operably linked to a therapeutic product to alleviate the symptoms or complications of said disease, or condition.

“Chronic” administration refers to administration of an electrical stimulus in a continuous mode as opposed to an acute mode, so as to maintain the initial therapeutic effect (activity) for an extended period of time.

“Electrical pulse generator” is a medical device that has the essential feature of being capable of providing an electrical stimulus or series of electrical stimulations or pulses (pacing). As illustrated herein, an electrical pulse generator is operatively coupled to provide at least one effective electrical stimulus or pulse to induce transcription of an electrical responsive promoter.

“Intermittent” administration is treatment that is not consecutively done without interruption and is repeated in the course of time.

“Ischemia” is defined as an insufficient supply of blood to a specific organ or tissue. A consequence of decreased blood supply is an inadequate supply of oxygen to the organ or tissue (hypoxia). Prolonged hypoxia may result in injury to the affected organ or tissue. “Anoxia” refers to a virtually complete absence of oxygen in the organ or tissue, which, if prolonged, may result in death of the organ or tissue.

“Hypoxic condition” is defined as a condition under which a particular organ or tissue receives an inadequate supply of oxygen.

“Anoxic condition” refers to a condition under which the supply of oxygen to a particular organ or tissue is cut off.

“Reperfusion” refers to the resumption of blood flow in a tissue following a period of ischemia.

“Ischemic injury” refers to cellular and/or molecular damage to an organ or tissue as a result of a period of ischemia and/or ischemia followed by reperfusion.

The term “patient” as used herein refers to any mammal, including humans, domestic and farm animals, and zoo, sports, or pet animals, such as cattle (e.g., cows), horses, dogs, sheep, pigs, rabbits, goats, cats, and non-domesticated animals such as mice and rats. In a preferred embodiment of the invention, the mammal is a human or mouse.

Administration “in combination with” one or more further therapeutic agents includes simultaneous (concurrent) and consecutive administration in any order.

A “therapeutically effective amount” is the minimal amount of electrical stimulation that is necessary to impart a therapeutic benefit or a desired biological effect to a patient. For example, a “therapeutically effective amount” for a patient suffering or prone to suffering or being prevented from suffering a disease from a disease is such an amount which induces, ameliorates, or otherwise causes an improvement in the pathological symptoms, disease progression, physiological conditions associated with, or resistance to succumbing to a disorder principally characterized by an increase in transcription of a therapeutic product. For example, a “therapeutically effective stimulus” is the amount of electrical stimulation necessary to express a therapeutically effective amount of a gene sequence or protein in an amount to provide a therapeutic benefit.

The term “pace” as used herein is the act of issuing an electrical stimulus delivered to the cellular tissue delivered from a pulse generator.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLUTRONICS™.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Pharmacologically effective stimulus” or “physiologically effective stimulus” is the amount of stimulus needed to provide a desired level of a therapeutic product in the patient to be treated to give an anticipated physiological response when the ERP is stimulated or paced. The precise amount of stimulation or pacing needed will depend upon numerous factors, e.g., such as the specific activity of the product, the delivery stimulus employed, physical characteristics of the product, its intended use, and patient considerations. These determinations can readily be determined by one skilled in the art, based upon the information provided herein. A “pharmacologically effective stimulus” means an amount of stimulation provided to an ERP that is capable of producing therapeutic levels of the product in a patient.

The term “administer an electrical stimulus” means to deliver electrical stimulation to a tissue. As applied in the present invention, the electrical stimulus is delivered to the tissue to regulate transcription of ERP promoters.

It is intended that the use of the term “product” is meant to encompass the production of proteins and nucleic acid. The resultant products function in primary or secondary cells to produce the desired therapeutic result.

“Threshold” or “subthreshold” stimulation refers to a relative level of applied stimulation. While “threshold” stimulation refers to a level of stimulation to evoke a further electrical or mechanical response in the excited tissue, e.g. the minimum electrical stimulus needed to consistently elicit a cardiac depolarization that can be expressed in terms of amplitude (volts, milliamps) and pulse width (milliseconds (msec)), or energy (microjoules). Subthreshold stimulation refers to the application of electrical stimulation to tissue at levels low enough not to elicit a gross electrical or mechanical response from the tissue, such as to not cause cardiac depolarization or muscle contraction. A subthreshold stimulus can be achieved by keeping either the amplitude or the duration of the electrical pulses below the threshold response levels for gross motor or nerve responses. This scheme allows one to deliver electrical stimulation to the tissue to induce a response from the electrically responsive promoter without having the unwanted side effects due to the stimulation of nerve or muscle cells, such as unwanted contraction and or uncomfortable tactile sensations, and the like. It is recognized that the present invention can be practiced by delivery of a threshold or subthreshold stimulus.

As used herein, the term “primary cell” includes cells present in a suspension of cells isolated from a vertebrate tissue source and cultured, or it can refer to the cells that reside in the tissue of the vertebrate that have not been removed. Primary cells are one potential source of genetically engineered cells.

Description

Details of Genetic Elements

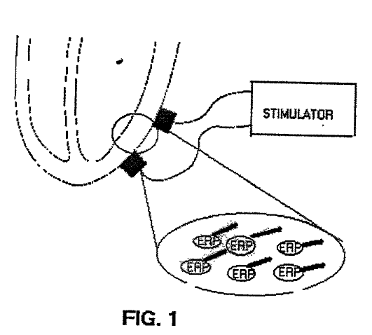

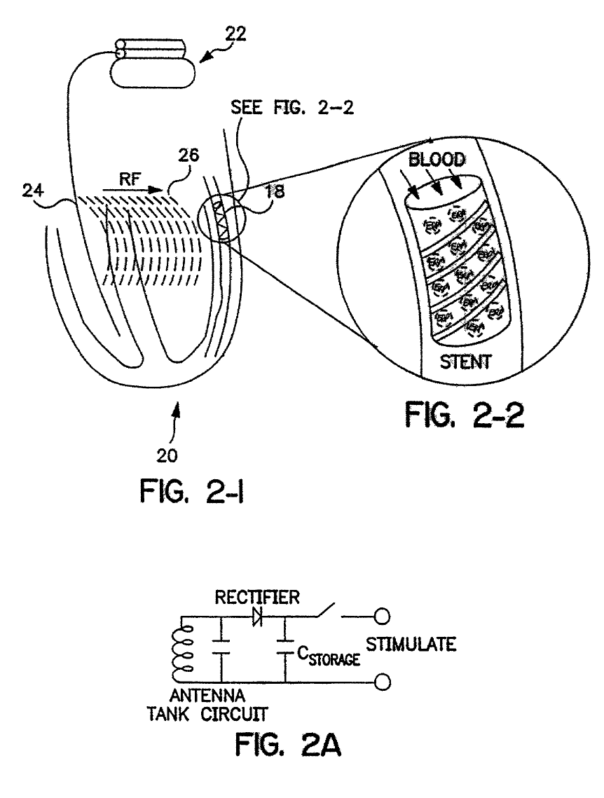

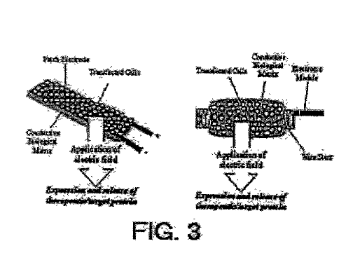

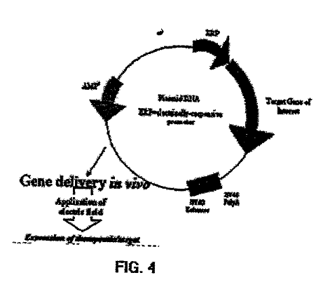

The present invention provides methods and systems for regulating delivery of therapeutic proteins and nucleic acids. Specifically, this involves using a genetically engineered electrically responsive promoter operably linked to a therapeutic gene sequence, wherein expression of said sequence is controlled by an electrical pulse generator (see FIGS. 1-3).

The present invention also provides chimeric genes having at least three functional elements: (i) a therapeutic gene, (ii) a promoter, and (iii) an electrical responsive enhancer (ERE) element, wherein the ERE is heterologous to at least one of the other functional elements. Optionally, other response elements (e.g., silencers, tissue specific elements, or enhancers) can be used in combination with the ERE element to direct expression of the therapeutic gene in a selected tissue when an appropriate electrical stimulus is given.

Promoters

A promoter, in the context of the present specification, refers to a polynucleotide element capable of promoting the transcription of a gene adjacent and downstream (3′) of the promoter. The promoter may contain all of, or only a portion of, the complete 5′ regulatory sequences of the gene from which it is derived. A sequence in the promoter region is typically recognized by RNA polymerase molecules that start RNA synthesis.

A promoter may be functional in a variety of tissue types and in several different species of organisms, or its function may be restricted to a particular species and/or a particular tissue. Further, a promoter may be constitutively active, or it may be selective for particular tissue types (e.g., a tissue specific element), or responsive to certain physiological conditions (e.g., hypoxia), or responsive to certain cell developmental stages (e.g., stem versus differentiated cell).

ERP Promoters

As previously defined, an “electrically responsive promoter” or “ERP” is a promoter that contains a genetically engineered electrically responsive element (ERE) that modulates transcription of the promoter in a cell upon the delivery of an electrical stimulus. At least one ERE may be operably linked to a given promoter, but a greater number of EREs may be used; 2, 3, 4, or more EREs may be operably linked. Modulated transcription may be positive or negative, and may change the relative transcriptional amount over time by an amount that is equal to or approximately 2, 4, 6, 10, 20, 50, 100, or 1000 fold or greater than unstimulated cells over 1, 2, 4, 8, 16, 24, 48, or 72 hours..

Generally one or more EREs is placed 5′ to the promoter at a position of approximately 20 to 30 bases upstream, 30-40 bases upstream, 40-60 bases upstream; 60-90 bases upstream; 90 to 150 bases upstream; 150-300 bases upstream; 300-600 bases upstream; and greater than 600 bases upstream from the site transcription initiation site. Determining the optimal place of responsive elements and determining the effect on transcription is well known to those skilled in the art. The level of expression of a gene under the control of a particular promoter can be modulated by manipulating the promoter region in relation to the different transcriptional elements. For example, different domains within a promoter region may be characterized by different gene-regulatory activities. The roles of these different regions are typically assessed using vector constructs having different variants of the promoter with specific regions deleted (i.e., deletion analysis). Vectors used for such experiments typically contain a reporter gene, which is used to determine the activity of each promoter variant under different conditions. Application of such a deletion analysis enables the identification of promoter sequences containing desirable activities. This approach may be used to identify, for example, the smallest region capable of conferring tissue specificity, or the smallest region conferring hypoxia sensitivity.

The present invention demonstrates contructions of the atrial natriuretic factor promoter that are electrically responsive (SEQ ID NO:1). Several ERP promoters have been identified as responsive to electrical stimulation that can also be suitably employed and practiced with the further teachings herein: ANF promoter (Sprinkle, A. B., et al., (1995); McDonough, P. M., et al., (1992);

McDonough, P. M., et al., (1994); McDonough, P. M., et al., (1997)); VEGF promoter (Annex, B. H., et al., (1998); Hang, J., et al., (1995), Kanno (1999)); acetylcholine receptor (Bessereau, J-L et al., (1994)); troponin I (Calvo, S., et al., (1996)); IGF-II (Fitzsimmons, R. J., et al., (1992)); NOS3 (Kaye, D. M., et al., (1996)); MCAD (Cresi, S., et al., (1996)); cytochrome c (Cresi, S., et al., (1996), Xia, Y., et al., (1998)); COX (Xia, Y., et al., (1997)); CPT-I (Xia, Y., et al., (1996)); hsp70(Yanagid, Y., et al., (2000)); skm2 (Zhang, H., et al., (1999)).

It is recognized that particular transcription factors, although not being bound by any particular mechanism of electrical stimulation, may be involved in enhancing transcription through EREs. These factors include, but are not limited to, NFAT3 (Xia, Y., et al., (2000)); GATA4 (Xia, Y., et al., (2000)); MEF2 (Calvo, S., et al., (1996); Mao, Z., et al., (1999)); c-Myc (Lin, H., et al., (1994)); cJun N-terminal kinase (McDonough, P. M., et al., (1997)); cJun SRF (McDonough, P. M., et al., (1997)); SP1 (Zhang, H., et al., (1999). McDonough, P. M., et al., (1997), Sprinkle, A. B., et al., (1993)); BDNF (Tabuchi, A., et al., (2000)); JunB (Xia, Y., et al., (1997)); NRF-1 (Xia, Y., et al., (1997)); AP1 (Xia, Y., et al., (1997)); CRE-1 (Xia, Y., et al., (1997)).

Tissue Specific Elements

Electrically responsive promoters useful in the practice of the present invention are preferably tissue specific—that is, they are capable of driving transcription of a gene in one tissue while remaining largely “silent” in other tissue types. It will be understood, however, that tissue specific promoters may have a detectable amount of “background” or “base” activity in those tissues where they are silent. The degree to which a promoter is selectively activated in a target tissue can be expressed as a selectivity ratio (activity in a target tissue/activity in a control tissue). In this regard, a tissue specific promoter useful in the practice of the present invention typically has a selectivity ratio of greater than about 2, preferably about 5 and even more preferably, the selectivity ratio is greater than about 15.

It will be further understood that certain promoters, while not restricted in activity to a single tissue type, may nevertheless show selectivity in that they may be active in one group of tissues, and less active or silent in another group. Such promoters are also termed “tissue specific”, and are contemplated for use with the present invention. For example, promoters that are active in a variety of central nervous system (CNS) neurons may be therapeutically useful in protecting against damage due to stroke, which may affect any of a number of different regions of the brain. In one application, electrically responsive promoters would be useful in the controlled production and release of enkephalins in the brain. Controlled production of enkephalins would be useful in pain management. Other uses in electrically responsive promoters would be the controlled production of natural dopamine agonist and antagonists by coupling expression of the natural analogs or their receptors, such as the D3 receptor, to electrically responsive promoters. Other relevant neural proteins or neurotrophic factors that would be therapeutically are BDNF, TNB, GDNF, NGF (nerve growth factor) and one or more of the capases (e.g., capase 1-9). Ideally, neurologic factors would be produced from neural cells. Neural cells may be transfected in vivo or ex-vivo with the relevant gene under control of an electrically responsive promoter. Where neural cells are transfected ex-vivo they are then transplanted into the desired site in the neural tissue. Within the range of transplanted neural cells, include mature neuronal cells, glial cells (e.g., astrocytes, oligodendrocytes), as well as neural stem cells and the like.

Other tissue specific promoters may be derived, for example, from promoter regions of genes that are differentially expressed in different tissues. For example, a variety of promoters have been identified which are suitable for upregulating expression in cardiac tissue. Included are the cardiac alpha-myosin heavy chain (AMHC) promoter and the cardiac alpha-actin promoter. Suitable kidney-specific promoters include the renin promoter. Suitable brain-specific promoters include the aldolase C promoter and the tyrosine hydroxylase promoter. Suitable vascular endothelium-specific promoters include the Et-1 promoter and vonWillebrand factor promoter.

A number of tissue specific promoters, described below, may be particularly advantageous in practicing the present invention. Tissue specific promoters are understood to relate to functional promoters that have a tissue specific element. In most instances, these promoters may be isolated as convenient restriction digest fragments suitable for cloning into a selected vector. Alternatively, promoter fragments may be isolated using the polymerase chain reaction (PCR) (U.S. Pat. No. 4,683,195). Cloning of amplified fragments may be facilitated by incorporating restriction sites at the 5′ ends of the primers.

Examples of tissue specifc promoters suitable for cardiac-specific expression include the promoter from the murine cardiac alpha-myosin heavy chain gene. The gene contains a 5.5 kbp promoter region which may be obtained as a 5.5 kbp SacI/SalI fragment from the murine alpha-MHC gene (Subramaniam, A., et al., (1993)). Reporter gene constructs utilizing this 5.5 kbp alpha-MHC promoter are expressed at relatively high levels selectively in cardiac tissue (Subramaniam, A., et al., (1993)). A smaller fragment of the rat alpha-MHC promoter may be obtained as a 1.2 kbp EcoRI/HindIII fragment (Gustafson, T., A., et al., (1987)). An 86 bp fragment of the rat alpha-MHC promoter, SEQ ID NO:2, restricts expression of reporter genes to cardiac and skeletal muscle (U.S. Pat. No. 5,834,306). Additional cardiac specificity may be conferred to the fragment by ligating (e.g., blunt end ligating) a 35-mer oligonucleotide (SEQ ID NO:3) containing cardiac-specific GATA4 enhancer elements just upstream of base pair −86 (Molkentin, J. D., et al., (1984)). This promoter fragment also results in low levels of expression in the absence of additional enhancers.

The sequences of exemplary cardiac-specific promoter regions from the rat and mouse AMHC genes are presented herein as SEQ ID NO:4 and SEQ ID NO:5, respectively. Both sequences end just upstream of the ATG initiation codons of their respective genes. Other cardiac-specific promoters include the cardiac alpha-actin promoter (a 118 bp fragment (SEQ ID NO:6) obtained from the human cardiac alpha-actin (HCA) promoter), and the cardiac-specific myosin light chain-2 promoter (a 2.1 kbp KpnI/EcoRI fragment from the rat cardiac myosin light chain-2 (MLC-2) gene (Franz, W-M. et al. (1993)).

Other tissue specific promoters known in the art can be adapted to incorporate ER elements. Prostate specific promoters include the 5′-flanking regions of the human glandular kallikrein-1 (hKLK2) gene and the prostate specific antigen (hKLK3; PSA) gene (Murtha, P. et al. (1993); Luke, M. C., et al. (1994)). The renin promoter is suitable for directing kidney specific expression (Fukamizu, A., et al., (1994)), while the aldolase-C promoter (Vibert, M., et al., (1989)) or the tyrosine hydroxylase promoter (Sasaoka, T., et al., (1992)) may be used to direct expression in the brain. Promoters specific for vascular endothelium cells include the Et-1 promoter (Inoue, A., et al., (1989)) and vonWillebrand factor (Jahrondi, N., et al. (1994)) promoter. Tumor specific promoters include the alpha-fetoprotein (AFP) promoter, contained in a 7.6 kbp fragment of 5′-flanking DNA from the mouse AFP gene (Marci, P., et al., (1994)). This promoter normally directs expression of the AFP gene in fetal liver and is transcriptionally silent in adult tissues. However, it can be abnormally reactivated in hepatocellular carcinoma (HCC), conferring tumor specific expression in adult tissue (Marci, P., et al. (1994)).

The above promoters are exemplary promoters for use with the present invention. Other promoters suitable for use with the present invention may be selected by one of ordinary skill in the art following the guidance of the present specification.

Plasmid Reporter Constructs

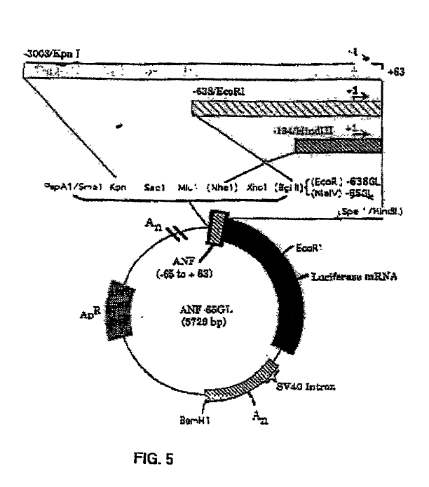

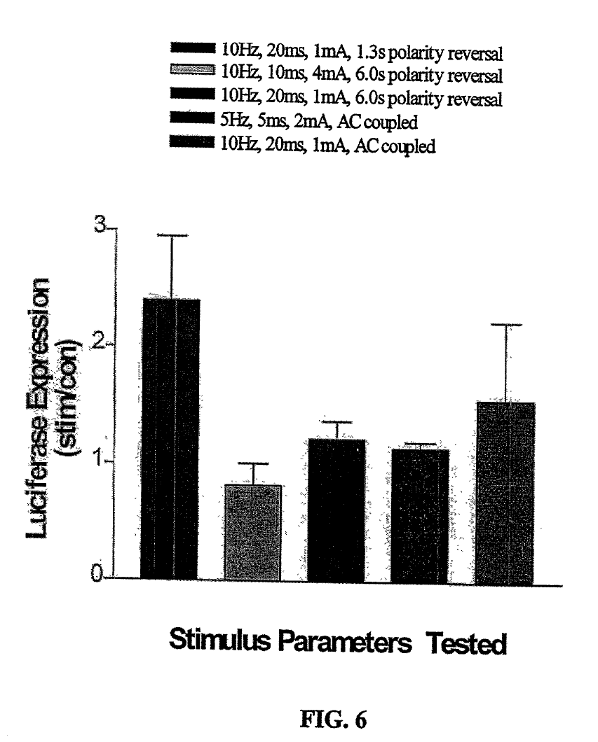

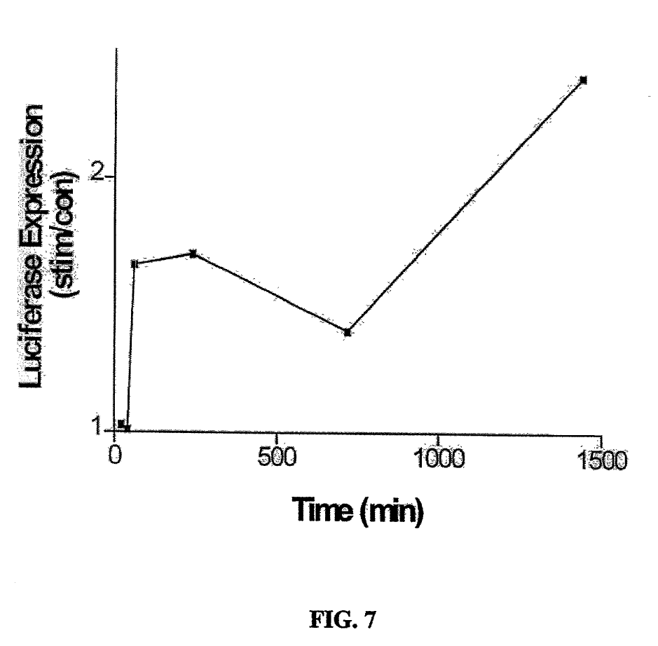

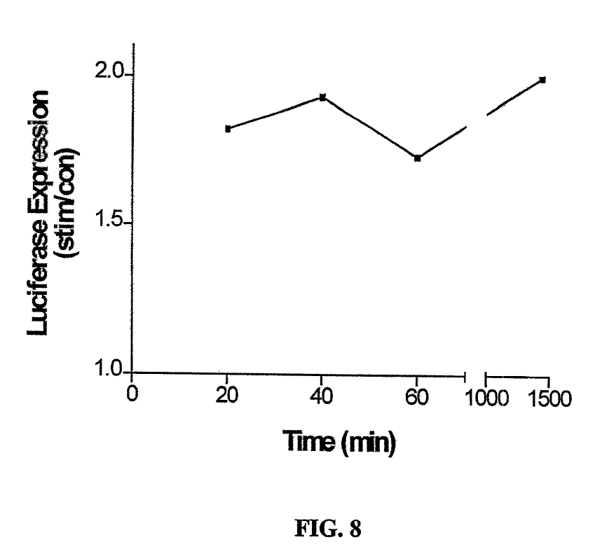

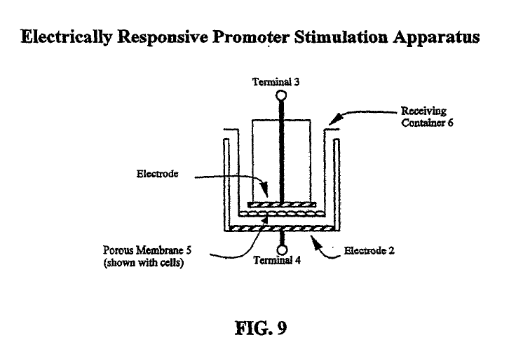

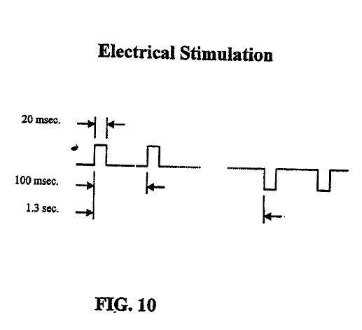

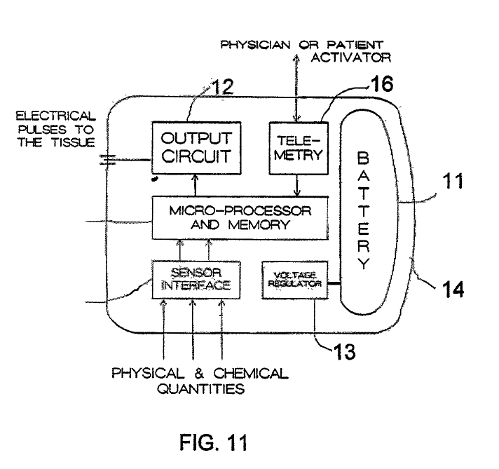

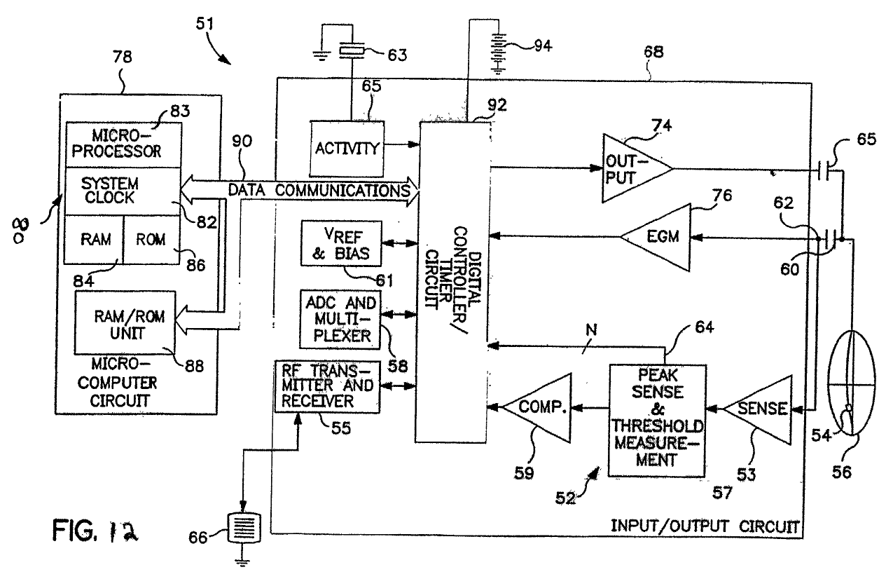

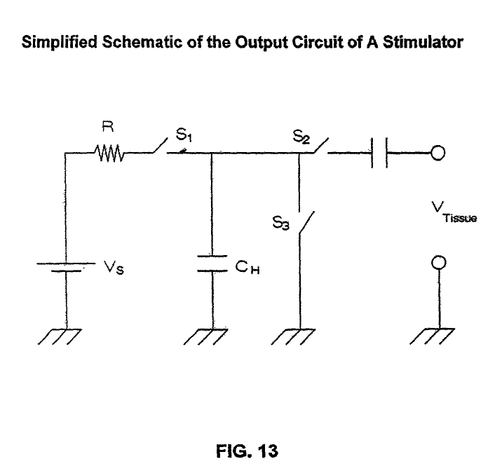

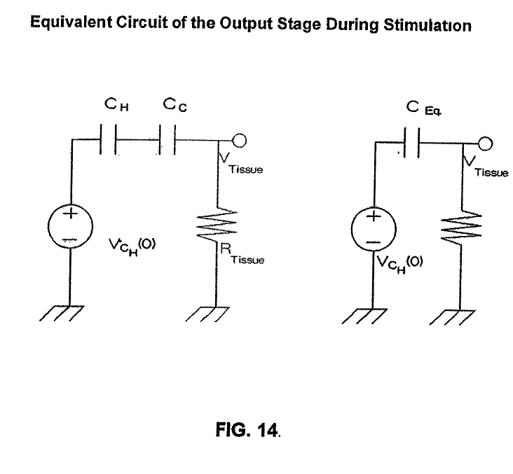

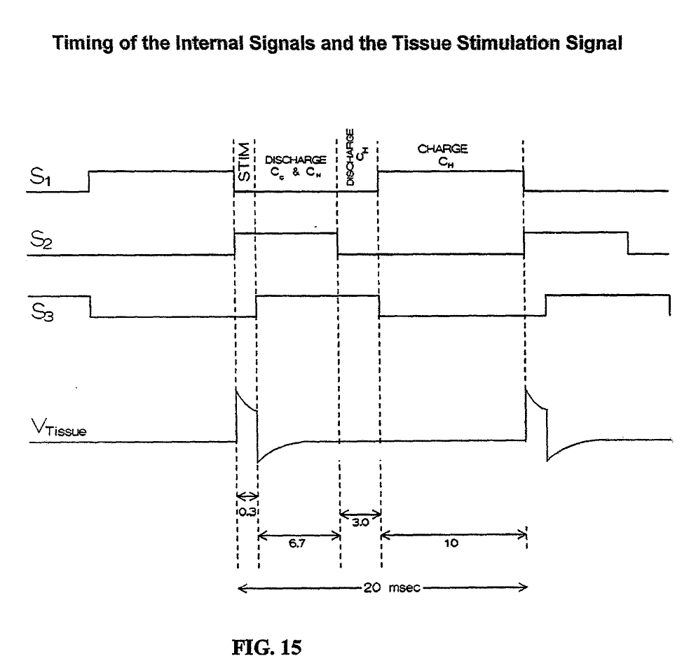

ANF 5′ flank/Luciferase reporter vectors were designated either −3003LUC, −638LUC, etc. (various truncations of the ANF 5′ flanking region of SEQ ID NO:1), or ANF-3003GL, ANF -638GL. The construction of the latter vectors is shown in FIG. 5; the vectors were created as follows. A Kpnl/SpeI fragment of the plasmid pANF 3003 (Knowlton, K. U., et al. (1991)) was cloned into the Kpnl/Hind III sites of pGeneLight2-Promoter (pGL2-P, Promega, Madison, Wis.), replacing the SV-40 promoter of the vector with rat ANF 5′ flanking sequences (FS) from −3003 to +65, to produce ANF-3003GL. Similar truncations were produced with HindIII/SpeI (ANF-134GL), EcoRI/SpeI (ANF-638GL) and NlaIVISpeI (ANF-65GL) fragments. The NlaIVISpeI fragment was inserted into pGL2-P utilizing BglII(filled)/HindIII sites, which allowed the use of the multiple cloning site for enhancer insertions upstream of the minimal ANF promoter. An internal deletion of the full length ANF flanking region was created by eliminating the HindIII fragment −691 to −134. Further truncations between −134 and −65 were created using −3003ANFGL digested with KpnI and HindlII as the starting template and using the Promega Erase-a-base kit according to the manufacturer's instructions. Site directed mutagenesis of ANF-134GL and ANF-638GL was performed using the Promega Altered Sites in vitro mutagenesis kit according to the manufacturer's instructions. All plasmid constructions were verified by restriction mapping and dideoxy sequencing.

Cells

A number of suitable permanent cell lines can be used and tested with transfected ERPs. Clones of these cell lines can be obtained from the American Tissue Type Collection. Preferred cell lines include QBI-293A, C2C12 cells, NIH-3T3, NG108, P19, and the like.

Primary cells from vertebrate tissue are isolated using known procedures, such as punch biopsy or other surgical methods. For example, punch biopsy is used to obtain skin as a source of fibroblasts or keratinocytes. A mixture of primary cells is obtained from the tissue using known methods, such as enzymatic digestion or explanting. If enzymatic digestion is used, enzymes such as collagenase, hyaluronidase, dispase, pronase, trypsin, elastase and chymotrypsin can be used in conjunction with known methods of isolation.

In one aspect the invention uses transplanted or grafted cells to introduce the electrically responsive system into a tissue. Transplanted or grafted cells for heart tissue can be chosen from the group consisting of: adult cardiomyocytes, pediatric cardiomyocytes, fetal cardiomyocytes, adult fibroblasts, fetal fibroblasts, adult smooth muscle cells, fetal smooth muscle cells, endothelial cells, and skeletal myoblasts.

Transplanted cells or grafts may be derived from auto-, allo- or xeno-graphic sources. Further, transplanted cells may comprise a suitable biodegradable or non-biodegradable scaffolding having cells supported thereon. A number of procedures are known in the art for isolating various primary cell types. For example see U.S. Pat. No. 6,099,832 and procedures described herein for isolation of adult cardiomyocytes, pediatric cardiomyocytes, fetal cardiomyocytes, adult fibroblasts, fetal fibroblasts, adult smooth muscle cells, fetal smooth muscle cells, endothelial cells, and skeletal myoblasts.

Ex Vivo Construction of ERP Cells

ERPs can be introduced into a wide variety of cells. As described herein, applicants have demonstrated that ERPs can be introduced into primary and secondary cells of mammalian origin and that ERP promoters can be stably integrated and operably linked to an exogenous genes using a wide variety of vectors.

Primary cells can be transfected directly or can be cultured first before transfection. Primary cells are transfected with exogenous ERP DNA operably linked to a gene sequence or the ERP DNA can be joined to appropriate flanking DNA sequences to properly direct its integration to the host gene sequence such that the exogenous ERP and host gene are operably linked. Optionally, DNA encoding a selectable marker is provided with the ERP DNA or the selectable marker is co-transfected with the ERP DNA.

One method of introducing the ERP DNA into the desired cell is by electroporation. Electroporation can be carried out over a wide range of voltages (e.g., 50 to 2000 volts) and corresponding capacitances. Total DNA of approximately 0.1 to 500 ug is generally used. Alternatively, ERP DNA can be introduced into cells using microinjection, calcium phosphate precipitation, modified calcium phosphate precipitation, polybrene precipitation, liposome fusion, receptor-mediated gene delivery, and the like. If the transfection is done ex vivo (herein referring to cells transfected outside the body of the patient), stably transfected cells are isolated and cultured and subcultivated under appropriate culturing conditions. Alternatively, more than one transfected cell is cultured and subcultured, resulting in production of a heterogeneous cell strain.

Further, the present invention is intended to cover the incorporation of an exogenous ERP that promotes the expression of a gene existing in the genomic DNA of a host, as described by U.S. Pat. No. 6,063,630. The ERP promoters or EREs can be incorporated into the endogenous cells of the host tissue, or primary cultured cell taken from the tissue, or in known cell lines. The exogenous ERP promoter is placed such that it can direct transcription of a therapeutic product, such as a therapeutic protein or RNA, to be expressed in the tissue cells or cultured cells. Homologous insertion of the EREs is such that they are placed relative to the endogenous promoter so that the natural promoter becomes responsive to electrical stimulation.

The number of cells needed to transfect a primary or clonal cell line depends on a variety of factors, including, but not limited to, the use of the transfected cells, the functional level of ERP expressed product in the transfected cells, the site of implantation of the transfected cells, and the age, surface area, and clinical condition of the patient. For example, to correct a myocardial infarction in a patient, approximately one million to five hundred million transfected myoblasts, more preferably approximately ten million to fifty million myoblasts, and most preferably approximately fifty million myoblasts are used.

Therapeutic Products

ERPs used (or identified by the procedures taught herein) in the course of the present invention have wide applicability as part of the present delivery system for a wide range of therapeutic products, such as enzymes, hormones, cytokines, antigens, antibodies, clotting factors, anti-sense RNA, regulatory proteins, transcription proteins and nucleic acid products, and engineered DNA. For example, the ERP can be used to supply a therapeutic protein, including, but not limited to, VEGF, nitric oxide synthetase, tissue plasminogen activators, Factor VIII, Factor IX, erythropoietin, alpha-1 antitrypsin, calcitonin, glucoscerebrosidase, growth hormone, low density lipoprotein (LDL) receptor, IL-2 receptor, insulin, globin, immunoglobulins, catalytic antibodies, interleukins, insulin-like growth factors, superoxide dismutase, immune responder modifiers, parathyroid hormone, interferons, nerve growth factors, and colony stimulating factors.

The wide variety of delivered therapeutics can be further categorized by products containing a secreted protein with predominantly systemic effects, a secreted protein with predominantly local effects, a membrane protein imparting new or enhanced cellular responsiveness, a membrane protein facilitating removal of a toxic product, a membrane protein marking or targeting a cell, an intracellular protein, an intracellular protein directly affecting gene expression, an intracellular protein with autolytic effects, gene product-engineered DNA which binds to or sequesters a regulatory protein, a ribozyme, or antisense-engineered RNA to inhibit gene expression.

Therapies

In one feature of the invention, the present system can be used to treat peripheral arterial occlusive disease (PAOD) or coronary arterial disease (CAD) or stroke, by delivery of therapeutically relevant genes. It is envisioned that treatment of peripheral arterial occlusive disease (PAOD) or coronary arterial disease (CAD) is achieved by the delivery of angiogenic proteins, such as VEGF and FGF, whereby delivery of the angiogenic proteins are used to enhance local blood vessel formation. In another aspect of therapy, the treatment of heart attack or stroke may be able to more effectively be treated by local delivery of tissue plasminogen activator (tPA).

In its simplest mode, to stimulate the electrically responsive elements within the cells of a patient, one would simply turn on the stimulating device. Programming would be required to be sure the amplitude of the electrical stimulation was sufficient to be turning on the gene. The appropriate amplitude would be determined as the lowest amplitude (or 2×, 3×, 4× or 5× the lowest amplitude, or as the case may be) that elicits a therapeutic outcome. In the absence of a detectable therapeutic result, the pacing amplitude would have to be set using an assay for the generated protein, or empirically using in vitro data on the amplitude versus distance from the cell to affect stimulation.

Administering Cells to a Patient

The genetically engineered cells containing an ERP may be introduced into a patient using known methods. The recombinant ERP cells produced as described above are introduced into an individual to whom the therapeutic product is to be delivered, using known methods, using various routes of administration (e.g., direct injection, injection through a catheter) and at various sites. In one feature of the present invention cells are delivered to muscular tissue of the heart or skeletal muscle, renal tissues, bone tissues, intestinal tissues, nerve tissues, hepatic tissues, dermal tissues, epidermal tissues, or the like. In another feature genetic material is directly introduced into cells of muscular tissue of the heart or skeletal muscle, renal tissues, bone tissues, intestinal tissues, nerve tissues, hepatic tissues, dermal tissues, epidermal tissues, or the like. Once implanted in the individual, the transfected cells are operably coupled to the electrical pulse generator. Generally this is accomplished by the implantation of electrodes and leads for carrying the electrical stimulus from the electrical pulse generator (FIG. 1 represents the system used in the heart).

In one aspect transfected primary or cultured ERP cells are used to administer therapeutic products by cell transplantation when delivered with conjunctive electro-stimulatory therapy. An advantage to the use of ERP transfected primary or cultured cells of the present invention is that the number of cells and location of their delivery can be controlled. Further, delivery of the therapeutic product can be controlled by the location of electrodes and the period of electrical stimulation.

The gene, or portions thereof, may be introduced into a target tissue as part of a complete expression vector in a pharmaceutically-acceptable carrier, either by direct administration to the target tissue (e.g., injection into the target tissue), or by systemic administration (e.g., intravenous injection). In the latter case, the gene may be targeted to a selected tissue, for example, by incorporating it in a virion expressing a modified envelope protein designed to bind to receptors preferentially expressed on cells from the selected, or targeted, tissue. Alternatively, the ERE has been introduced into a tissue compatible cell type which is then transplanted into the targeted tissue in a pharmaceutically-acceptable carrier by direct administration. In select cases the ERP cells may be delivered systemically. As further described herein, a variety of therapeutic genes, promoters, and EREs may be employed in the practice of the present invention.

Introducing ERP DNA In Vivo into Patient Cells

Several types of viruses, including retroviruses, adeno-associated virus (AAV), may be amenable for use as vectors with chimeric gene constructs of the present invention. Each type of virus has specific advantages and disadvantages, which are appreciated by those of skill in the art. Methods for manipulating viral vectors are also known in the art (e.g., Grunhaus and Horowitz; Hertz and Gerard; and Rosenfeld, et al.). Alternatively, DNA may be directly injected into the target tissue.

Retroviruses, like adeno-associated viruses, stably integrate their DNA into the chromosomal DNA of the target cell. Unlike AAV, however, retroviruses typically require replication of the target cells in order for proviral integration to occur. Accordingly, successful gene transfer with retroviral vectors depends on the ability to at least transiently induce proliferation of the target cells.

Adeno-associated viruses are capable of efficiently infecting nondividing cells and expressing large amounts of gene product. Furthermore, the virus particle is relatively stable and amenable to purification and concentration. Replication-defective adenoviruses lacking portions of the E1 region of the viral genome may be propagated by growth in cells engineered to express the E1 genes (Jones and Shenk; Berkner; Graham and Prevea). Most of the currently-used adenovirus vectors carry deletions in the E1A-E1B and E3 regions of the viral genome. A number of preclinical studies using adenoviral vectors have demonstrated that the vectors are efficient at transforming significant fractions of cells in vivo, and that vector-mediated gene expression can persist for significant periods of time (Rosenfeld, et al.; Quantin, et al.; Stratford-Perricaudet, et al., 1992a; Rosenfeld, et al.; L. D. Stratford-Perricaudet, et al., 1992b; Jaffe, et al.). Several studies describe the effectiveness of adenovirus-mediated gene transfer to cardiac myocytes (Kass-Eisler, et al.; Kirshenbaum, et al.).

One approach to delivering ERPs operably linked to therapeutic genes utilizes adenovirus replication deficient vectors for delivery to the desired tissue. One such vector is the AdenoQuest™ adenovirus expression system (Quantum Biotechnologies, Inc). This recombinant adenovirus can infect many different cell lines or tissues of human or non-human origin. The virus enters the cell but does not replicate. This abortive infection can be seen as a “transfection system” to introduce ERPs operably linked to genes.

Plasmids bearing chimeric genes of the present invention may be purified and injected directly into a target tissue. For example, direct injection of plasmid suspended in saline buffer is effective to result in expression of the plasmid in the cardiac cells. Similar approaches have been used successfully by others to express, for example, exogenous genes in rodent cardiac and skeletal muscle (Wolf, et al.; Ascadi, et al., 1991a; Ascadi, et al., 1991b; Lin, et al.; Kitsis, et al..

Liposomes may be employed to deliver genes to target tissues using methods known in the art. The liposomes may be constructed to contain a targeting moiety or ligand, such as an antigen, an antibody, or a virus on their surface to facilitate delivery to the appropriate tissue.

Details of Electrical Pulse Generators

Apparatus for Testing Cells

The main purpose of the test apparatus is to test any given promoter for its ability to be regulated by an applied electrical field. Using the constructed apparatus (FIG. 9), any given promoter or responsive element can be inserted into a reporter plasmid to test its responsiveness to electrical stimulation, or to determine the effect of placement of one or more EREs on transcription with other functional transcriptional sequences.

The test apparatus of FIG. 9 consists in part of a separable pair of plate electrodes 1 and 2 operably coupled to terminals 3 and 4 during operation. Terminals 3 and 4 operably couple to the pulse generator (not shown). Plate electrodes 3 and 4 serve to transmitted energy through a porous membrane 5. Porus membrane serves to support the testing material between electrodes 1 and 2, and to uniformly pass electrical current through the supported testing material and membrane.

The applicants have demonstrated that the use of p638 ANFluc (FIG. 5) in conjunction with the test apparatus provides a functional assay for testing for ERPs and determining the effect on the cell with any given electrical stimulation routine. As an example, that cells can be tested in the constructed apparatus, FIG. 10 shows one type of electrical stimulation (applied from terminal 3 to terminal 4) that resulted in a detected response of the Electrically Responsive Promoters (ERPs). This stimulation consists of a train of 20 msec. pulses at a rate of 10 Hz. (100 msec. from one pulse to the next). The pulses are monophasic (not charge balanced), but the polarity of the pulses is reversed every 1.3 secs. Other pulse forms can be used and tested in the described apparatus to test various conditions (amplitude (volts, milliamps) and pulse width (milliseconds (msec)), or energy (microjoules), or wave form (monophasic, biphasic, and the like)) of electrical stimulation on ERP driven expression.

The apparatus is designed to produce uniform electric fields spatially, so that all of the cells being tested experience the same electric field intensity. The parallel plates of the electrodes 1 and 2 in this apparatus produce a field of this type. One embodiment of this apparatus consists of an upper electrode 1 that is slightly smaller than the porous membrane, which in turn is slightly smaller than the lower electrode 2. In another embodiment, the electrodes 1 and 2 would be the same size, with the porous membrane 5 being slightly smaller than the two electrodes. This embodiment would minimize any electric field fringing effects that occur at the edge of the parallel plates. These fringing effects reduce the uniformity of the electric field. However, it is recognized a number of different sizes and shapes of the electrodes and membrane can be chosen.

One embodiment of this apparatus uses titanium as the electrode material; however, there are many other conductive materials that could be used, such as platinum, gold, silver, etc. Titanium or platinum electrodes have the advantage of low reactivity in ionic solutions; however, more reactive metals could be chosen depending on the type of electrical stimulation applied, the amount of buffering solution, etc.

One embodiment of this apparatus uses a solid electrode; however, there are other possible electrode configurations. In an alternative embodiment the material is formed into a mesh. This embodiment is particularly desirable if the electrode material is expensive (e.g., platinum or gold), since less of the material is needed to form the electrode. In another embodiment, as shown in FIG. 9, the lower electrode forms a receiving container for porous membrane 5 and the upper electrode 1. Likewise, the the porous membrane 5, can be fashioned to be part of a receiving container for the upper electrode 1.

In one embodiment of this apparatus, mammalian cells are placed on the porous membrane, which is placed between the electrode plates. The membranes are generally composed of a porous polymeric material, such as PET (polyethylene terephthalate). Generally, the pore size may vary any where between about 40 and 0.004 microns, preferably between about 4 and 0.04 microns, and most preferably a pore size of about 0.4 microns with a pore density of approximately 1,600,000 pores/square centimeter.

In other embodiments, alternative materials can be used in place of the mammalian cells and analyzed for their response to an electrical field. For example, enzymes with moieties that have a net electric charge would change their conformation based on the electric field intensity. This conformational change could affect the reaction rate of the enzyme. Thus, the effect of different types of electric fields on the reaction rates of some enzymes could be analyzed with this apparatus. In an alternative embodiment, electrical field can be applied without the use of the electrodes in contact with the tissue. In one embodiment, body can be subjected to alternating magnetic fields, oriented in a direction normal to the plane of cells. Electrical currents and fields circling the magnetic field vector will be induced due to the Faraday's Law of Induction. Intensity of these currents, also known as Eddy Currents, will have be proportional to the frequency of excitation and the strength of the magnetic field, but will diminish as the distance from the source of the magnetic field increases. This embodiment would be practical in the case where the cells containing ERP are close to the skin, and eliminates the need for an implantable stimulator and electrode system.

In another alternative embodiment, one can simply place parallel plates outside of the body, in contact with the skin or preferably not in contact with the skin, to induce displacement currents in the body. By periodic alteration of the polarity of the voltage applied to the plates, displacement charges would be swept across the body, producing the electrical stimulation needed for the ERP. This embodiment is preferred when the cells containing ERP are deep in the tissue. Again, this embodiment eliminates the requirement for the implantable stimulator and the electrodes.”

Electrical Pulse Generator

One essential element of the present invention is the provision of an electrical pulse generator. An electrical pulse generator has the essential feature of being capable of providing an electrical stimulus or series of electrical stimulations or pulses (pacing). The electrical stimulus or pulses can be used to induce transcription of an electrical responsive promoter. In one embodiment, the electrical stimulator provides a subthreshold stimulation to activate transcription of a therapeutic product. The objective of the subthreshold stimulation is not to excite the tissue for mechanical contraction but to selectively activate the synthesis of therapeutic products, e.g., enzymes, proteins, growth factors, or other biologically active substances, such as other nucleic acids or proteins that may regulate other biological activities. However, different stimulation patterns may be given in conjunction with other electrical stimulation therapies. At times, particularly when considered with other electrical stimulatory therapies, threshold electrical stimulation may be given or may be advantageous.

The controlled output voltage from the electrical pulse generator can be adjusted for a wide range of issue impedances, such as from 35Ω to infinity. The electrical pulse generator can be used to deliver subthreshold stimulation or threshold stimulation. In one embodiment, a subthreshold stimulus is provided wherein the stimulation device is able to deliver a charged balanced electrical pulse at a rate of 50 to 60 Hz, and at peak amplitudes of 0.1 volts. This combination of settings has been shown to evoke increased transcription.

One feature of the provided system is to allow the electrical pulse generator to have temporal control as well as spatial control of the ERP in vivo. Generally this is done to evoke a maximal ERP response with the given stimulus

It is envisioned that the electrical pulse generator can be implanted or can be external. Most often the stimulation is provided through a set of leads and electrodes from the pulse generator to the tissue cells containing an ERP.

Attaching lead and electrodes to the pulse generator are designed to stimulate transcription of at least one ERP. A number of suitable electrodes can function to provide the electrical stimulation to the tissue bearing ERPs. In one feature, the electrode is a surface coil electrode. The surface electrode may be constructed of a platinum alloy or other biocompatible metals. The electrode can be a coil, a cylinder, a wire, or any other shape.

The delivery system of the present invention includes a pulse generator (i.e., a stimulation device) that includes a stimulating element, such as a pulse generator (PG) similar in many respects to pacemakers and defibrillators known in the art. A pulse generator 22 shown in FIG. 11 contains an electrochemical cell (e.g.,battery 11) for providing electrical current to output circuit 12 that is controlled through voltage regulator 13. The pulse generator may include a hermetically sealed enclosure 14 that may include various elements, micro-processor and memory circuitry 15 that controls device operations, a telemetry element 16 that has a transceiver antenna, and a circuit that receives, stores, and transmits telemetry commands, and a sensing element 17 monitors the physical and chemical status of the patient.

If a telemetry element is employed, it contains a means of receiving and transmitting radio frequency commands and information between the device and the patient or physician in a manner that allows regulating the output of the pulse generator.

If a sensing element is employed, the sensing element monitors the patient to detect when a stimulus needs to be sent to the cells to trigger release of one or more therapeutic agents. This monitoring can be in the form of an electrocardiogram (ECG), for example, to detect an ST segment elevation or a reduction of blood flow in the coronary sinus. Once the sensing element detects a need to deliver a therapeutic product it signals the pulse generator to provide an electrical stimulus or set of electrical stimulations to the ERP promoters to transcribe the therapeutic gene. For example, when a blood clot is formed in the heart, it reduces blood flow and produces an abnormal ECG which is sensed and causes the PG to trigger ERP promoters to transcribe tPA, which is synthesized and excreted to reach the blood clot, thereby preventing or lessening the likelihood that the blood clot may lead to a myocardial infarction.

Threshold Stimulation

In one mode the pulse generators (PGs) are designed to stimulate cardiac muscle tissue; they may be modified readily by one of skill in the art to stimulate ERP-cells in accordance with the teachings of the present invention. It will be appreciated that the stimulation device according to the present invention can include a wide variety of microprocessor-based pulse generators similar to those used in pacemakers, as disclosed in U.S. Pat. Nos. 5,158,078 (Bennett et al.), 5,312,453 (Shelton et al.), and 5,144,949 (Olson), and pacemaker/cardioverter/defibrillators (PCDs), as disclosed in U.S. Pat. Nos. 5,545,186 (Olson et al.), 5,354,316 (Keimel), 5,314,430 (Bardy), 5,131,388 (Pless), and 4,821,723 (Baker et al.). Alternatively, the pulse generator device can include stimulating elements similar to those used in implantable nerve or muscle stimulators, such as those disclosed in U.S. Pat. Nos. 5,199,428 (Obel et al.), 5,207,218 (Carpentier et al.), and 5,330,507 (Schwartz).

FIG. 12 is a block diagram illustrating various components of an stimulation device 22, which is programmable by means of an external programming unit (not shown). One such programmer easily adaptable for the purposes of the present invention is the commercially available Medtronic Model 9790 programmer. The programmer is a microprocessor device which provides a series of encoded signals to stimulation device 22 by means of a programming head which transmits radio frequency encoded signals according to a telemetry system, such as that described in U.S. Pat. No. 5,312,453 (Wyborny et al.), for example. Stimulation device 22, illustratively shown in FIG. 12 as an exemplary embodiment, is electrically coupled to lead or antenna 24. Lead 24 may be used for stimulating only, or it may be used for both stimulating and sensing. Lead 24 is coupled to a node 62 in the circuitry of stimulation device 22 through input capacitor 60. Input/output circuit 68 also contains circuits for interfacing with stimulation device 22, antenna 66, and circuit 74 for application of stimulating signals to lead 24 under control of software-implemented algorithms in microcomputer unit 78.

Microcomputer unit 78 comprises on-board circuit 80, which includes system clock 82, microprocessor 83, and on-board RAM 84 and ROM 86. In this illustrative embodiment, off-board circuit 88 comprises a RAM/ROM unit. On-board circuit 80 and off-board circuit 88 are each coupled by a data communication bus 90 to digital controller/timer circuit 92. The electrical components shown in FIG. 12 are powered by an appropriate implantable battery power source 94 in accordance with common practice in the art. For purposes of clarity, the coupling of battery power to the various components of stimulating element 22 is not shown in the figures.

Antenna 66 is connected to input/output circuit 68 to permit uplink/downlink telemetry through RF transmitter and receiver unit 55. Unit 55 may correspond to the telemetry and program logic disclosed in U.S. Pat. No. 4,556,063 (Thompson et al.), or to that disclosed in the above-referenced Wyborny et al., patent. Voltage reference (VREF) and bias circuit 61 generates a stable voltage reference and bias current for the analog circuits of input/output circuit 68. Analog-to-digital converter (ADC) and multiplexer unit 58 digitizes analog signals and voltages to provide “real-time” telemetry signals and battery end-of-life (EOL) replacement functions.

Sense amplifier 53 amplifies sensed signals and provides an amplified signal to peak sense and threshold measurement circuitry 57. Circuitry 57, in turn, provides an indication of peak sensed voltages and measured sense amplifier threshold voltages on path 64 to digital controller/timer circuit 92. An amplified sense amplifier signal is then provided to comparator/threshold detector 59. Sense amplifier 53 may correspond in some respects to that disclosed in U.S. Pat. No. 4,379,459 (Stein).

Circuit 92 is further preferably coupled to electrogram (EGM) amplifier 76 for receiving amplified and processed signals sensed by an electrode disposed on lead 24. The electrogram signal provided by EGM amplifier 76 is employed when the implanted device is being interrogated by an external programmer (not shown) to transmit by uplink telemetry a representation of an analog electrogram of the patient's electrical heart activity. Such functionality is, for example, shown in previously referenced U.S. Pat. No. 4,556,063. Note that lead or antenna 24 may be located in positions other than inside the heart.

Output pulse generator 74 provides stimuli to lead 24 through coupling capacitor 65 in response to a stimulating trigger signal provided by digital controller/timer circuit 92. Output amplifier 74, for example, may correspond generally to the output amplifier disclosed in U.S. Pat. No. 4,476,868 (Thompson).

It is to be understood that FIG. 12 is an illustration of an exemplary type of stimulation device which may find application in the present invention, or which may be modified for use in the present invention by one of skill in the art, and is not intended to limit the scope of the present invention. Electrical stimulation can be delivered using a pulse generator, capable of producing electrical impulses with predetermined timing and wave shape. This implantable pulse generator has a power source that is a chemical battery to provide power to in-house electronics as well as to power the output circuitry to generate the electrical pulses to be delivered to the tissue. Optionally, in one feature the pulse generator would also contain a telemetry device that would allow it to be programmed by a physician and/or to be triggered by a patient activator to initiate the therapy resulting from the electrically responsive promoter. In an alternative embodiment, the stimulator could contain sensors to measure physiological parameters and biochemical agents that can be used to provide the input to the control algorithm, so that the implantable stimulator would autonomously initiate the therapy.

Subthreshold Stimulation

In one aspect, the present invention provides an electrical pulse generator that is capable of providing subthreshold stimulation (FIGS. 13 and 14) to the tissue containing engineered ERPs. Specifically, the pulse generator is able to deliver charge balanced electrical pulses at rate of about 10 to 100 Hz, more preferably about 30 to 80 Hz, and more preferably about 50 to 60 Hz. Preferably, peak amplitudes of stimulation are approximately 0.3 volts, and more preferably approximately 0.2 volts, and most preferably 0.1 volts. Preferred amplitudes of stimulation are such that they are below the stimulation threshold, i.e., subthreshold stimulation.

In one feature the present invention provides a pulse of 50 Hz; each pulse has a 0.3 msec of stimulus and a 6.7 msec of recharge with opposite polarity for charge balance, and electrodes are floating for the remainder of 13 msec of the pulse cycle. FIG. 15 shows the timing diagram of the electrical stimulation pulse, as well as the internal timing of the circuit providing this pulse train.

The schematic of the output circuitry FIG. 13 illustrates the schematic of the output circuitry for a subthreshold pulse generator is shown in FIG. 13. For example, the component values can be chosen as follows: VS=2.8Volts, R=25Ω, CC═CH=10 μF. One of these values are chosen such that the CH will have 0.110 volts at the end of 10 msec charging phase as shown in FIG. 15. By this illustration one skilled in the art could choose a number of settings that would provide CH at any given set voltage. FIG. 14 shows the equivalent circuit of the output stage during the stimulation phase. V_{C} represents the initial condition on the C_{H}. In this case C_{H}, C_{C} and Rₜᵢₛₛᵤₑ are in series connection. One can combine C_{H} and C_{C} into C_{eq}=5 μF. Voltage seen at the electrodes are given by: V_{Tissue}(t) =V_{CH}(0) {1−exp[−t/(C_{Eq}R_{Tissue})]} If, for example, the output voltage is allowed to change by only 10%, then the V_{Tissue}(t) will vary between 0.110 volts and 0.090 volts. That would indicate that V_{CH}(0)=0.110, and V_{Tissue}(t)(0.3 msec)=0.090. Rewriting the equation for the tissue voltage, 0.090=0.110 {1−exp[−t/(C_{Eq}R_{Tissue})]}, t=0.3 msec or 0.090=0.110 {1−exp[−0.3×10⁻³/(5×10⁻⁶×R_{Tissue})]} solving for R_{Tissue} one can find that R_{Tissue}=35Ω. In other words, the minimum tissue impedance that one can drive will be 35Ω, with output voltage staying in the 90-110 mV range. Use of the above settings of the pulse generator provides one example for (1) a pulse generator for subthreshold stimulation; (2) controlled output voltage for a wide range of tissue impedances (35Ω to ∞); (3) a pacing output for subthreshold stimulation where the objective is not to excite the tissue for mechanical contraction but to release therapeutic products from a ERP promoter; and (4) temporal control of the cellular machinery.

The accompanying lead system is to deliver this stimulation to selected tissue beds to derive maximal response from ERP promoters and to minimize undesirable cell stimulation such as cardiac muscle excitation/contraction, which could induce Vfib at 50 Hz.

Electrode placement can be done in one of two ways: In the preferred embodiment, electrodes are advanced to the vicinity of the tissue where the transfected cells are located, using the venous system, and left in place. Alternatively, it is possible to place the electrodes in place using minimally invasive surgical procedures, which would allow access to locations that are beyond the reach of the catheters in the vasculature. In either case, bipolar or unipolar stimulation can be applied to generate the electrical fields in the tissue to trigger the electrically responsive promoter. Bipolar stimulation is the preferred method.

The placement of the electrodes would be determined primarily by the method used to implant the electrodes. If the electrodes are placed via a transvenous route then the electrodes should be placed as close a possible to the implanted cells, understanding that the patient anatomy may not allow close proximity of the electrodes to the modified cells. If a non-transvenous implant technique is used then the stimulating electrodes can usually be placed very close to the modified cells. To minimize the energy used by the device to turn on the protein generation, the electrodes should be placed as close as possible to the modified cells.

Optional Sensing Element

In addition to a stimulating element within stimulation device 22, systems of the present invention may include a sensing element for monitoring at least one physiological property to detect a change in a physiological condition (typically, the onset of ischemia caused by a decrease in blood flow due to an occlusion resulting from the rupture of unstable plaque). For example, a pseudo-surface electrocardiogram (ECG) using a subcutaneous electrode array can be used to detect a reduction in blood flow, which is represented by an abnormal morphology (e.g., inverted shape) of a T wave (i.e., the portion of an ECG pattern due to ventricular repolarization or recovery). Such a pseudo-surface ECG is similar to a normal ECG modified for implantation. In this sensing element, for example, an implantable pulse generator having three electrodes, about one centimeter apart, could be implanted into the pectoralis muscle in the chest of a patient. An ECG pattern, similar to that of a normal ECG, would be monitored for an indication of an abnormal morphology of a T wave.

Sensing elements can include one or more individual sensors for monitoring one or more physiological properties. In addition to a pseudo-surface ECG, such sensors include, for example, blood gas (e.g., CO2) sensors, pH sensors, blood flow sensors in the coronary sinus, and the like. Other mechanisms of detection that can be used in sensors include, for example, acoustic time of flight changes as a result of flow, acoustic doppler, which takes advantage of the doppler effect (received frequency is different that the transmitted one), thermal dilution (a clinical technique to measure blood flow and cardiac output), and venous pressure drop due to lack of driving pressure from the blocked artery. Examples of sensors or implantable monitoring devices that can be modified for use in the stimulation devices of the present invention are disclosed, for example, in U.S. Pat. Nos. 5,409,009 (Olson), 5,702,427 (Ecker et al.), and 5,331,966 (Bennet et al.). Suitable sensors and sensing techniques are well known to one of skill in the art and can be readily adapted for use in the present invention.

### EXAMPLES

The present invention is further described by the following examples. The examples are provided solely to illustrate the invention by reference to specific embodiments. These exemplifications, while illustrating certain specific aspects of the invention, do not portray the limitations or circumscribe the scope of the invention.

Materials and Assays

The various restriction enzymes disclosed and described herein are commercially available and the manner of use of said enzymes including reaction conditions, cofactors, and other requirements for activity are well known to one of ordinary skill in the art. Reaction conditions for particular enzymes are carried out according to the manufacturer's recommendation.

We have utilized the Dual Luciferase Assay (DRL) to quantify the expression of luciferase in transfected cells. The protocol followed was essentially as desribed in the Promega product information data. β-Galactosidase: [does this protocol work for everything?]

Cells, cryosections, or tissue samples are fixed for 4 minutes at 4° C. in 4% paragormaldehyde, 0.25% glutaraldehyde, 100 mM NaH2PO4 (pH 7.4) before incubating for six hours at 37° C. in 1 mM 5-bromo-4-chloro-3-indolyl β-D-galactoside, 5 mM potassium ferricyanide, 5 mM potassium ferrocyanide, 2 mM MgCl2 in phosphate buffered saline (PBS). After wash with phosphate buffered saline (PBS) and counted in gelvatol, the samples are evaluated by microscopy.

### Example 1

Cell Cultures

QBI-293A (human kidney cell line, Quantum Biotechnologies) and C2C12 (mouse skeletal muscle myoblasts, ATCC) cell lines were cultured on 35 mm cell cultures inserts placed in 6-well plates (Falcon) according to vendor protocols. For gene transfection, cells were grown to approximately 60-80% confluency.

### Example 2

Cell Transfections

Cells were co-transfected using Fugene 6 (Roche Molecular Biochemicals) with either pGL2 (Clontech)/pRLSV40 (Clonetech) or p638ANFluc/pRLSV40 plasmid DNA. Each plasmid construct encoded a luciferase promoter gene fused to either an SV40 constitutive promoter (pGL2, pRLSV40) or a truncated atrial natriuretic factor promoter (p638ANFluc, FIG. 5. Briefly, 1-2 ug of each plasmid (4 ug total) was mixed with 15 ul of Fugene 6 and 85 ul of DMEM (Dulbeccom's Modified Eagle's Medium, Sigma Chemical Co.) growth medium, and the mixture was added drop wise to the cells. Cells were placed in a 37° C. humidified CO₂ incubator overnight, and then taken for electrical stimulation experiments.

### Example 3

Device for Testing Electrically Responsive Promoters

The present device described can be used to test whether any given promoter is responsive to electrical stimulation. The target promoter of interest is fused to a reporter gene sequence as described in Examples 1-2. Cell culture inserts with transfected cells are placed in the testing device, which is designed to evenly electrically stimulate the adherent cells.

Physical Description of the Apparatus:

The stimulation apparatus is based on a modified 6-well polystyrene cell culture plate. FIG. 9 is a schematic representation of one of the wells as viewed from the side.

In one feature the upper plate electrode 1 of the stimulation apparatus consists of a titanium disk attached to a polymer (Delron) cylinder, which is in turn attached to the cover of the cell culture plate. The upper electrode 1 is connected (with a titanium wire) to electrical terminal 3.

In another feature the lower plate electrode 2 consists of a titanium disk attached to the bottom of the well of the cell culture plate. The lower electrode is connected (with a titanium wire) to electrical terminal 4.

Cells to be stimulated by the apparatus are grown on the porous membrane 5. As tested the insert comprised a thin porous membrane attached to the base of a cell insert. When the cells were ready for stimulation, the cell attached to the membrane were placed in the well of the stimulation apparatus and the cover 8 (to which the upper electrode 1 is attached) is placed above the membrane with the cells. This results in the configuration shown in FIG. 9. The monolayer of cells is was suspended approximately 1 mm. from the lower electrode (terminal 2) and 2 mm. from the upper electrode (terminal 1).

The cells are surrounded by cell growth media which (because of its ionic content) is conductive. Since the membrane on which the cells are grown is porous, electrical stimulation applied from terminal 3 to terminal 4 is conducted through the attached membrane layer of cells.

Because the two electrodes are parallel disks separated by a small distance (approximately 3 mm.), the electrical field generated by the stimulation will be uniform across most of the cells. The exceptions are the small number of cells near the periphery of disks where fringing effects occur, resulting in a non-uniform electrical field.

FIG. 10 shows the type of electrical stimulation (applied from terminal A to terminal B) that results in the most optimum (maximum) response of the Electrically Responsive Promoter (ERP). This stimulation consists of a train of 20 msec. pulses at a rate of 10 Hz. (100 msec. from one pulse to the next). The pulses are monophasic (not charge balanced), but the polarity of the pulses is reversed every 1.3 secs. The pulse amplitude was determined by measuring the pulse current rather than the voltage. In the illustrated experiment, the optimal response of the ANF ERP (Electrically Responsive Promoter) in this apparatus was determined to be 2 mA. However, it is recognized the optimal settings are highly variable depending on the electrodes, the distance to the cell, and the electrode shape, size and configuration, as well cell density and cell type. Therefore, it is recognized a range of amplitudes can be determined for setting in-vivo performance parameters.

### Example 4

Testing for ERP Transcription

Human coronary artery endothelial cells (HCAEC) were electrically stimulated in the test chamber (FIG. 9) Cells were stimulated for 10, 20, and 60 minutes and then harvested 24 hours later. A time course study was also done where cells were harvested 8, 13, and 25 hours post stimulation. RNA was isolated from the harvested cells and reverse transcribed (RT) cDNA products from the RT reaction were quantified either by competitive PCR (tPA) or by semi-quantitatively PCR (bFGF, PDGF-B, TGF-1) with G3PDH as a control. tPA protein levels were also quantified by ELISA.

In the time course study, tPA expression levels increased up to 2.4 fold for the 8 and 13 hour post-stimulation time-points, and returned to near basal levels after 24 hours. Concomitant increases in tPA protein levels were seen at the 8 and 13 hour time points. In the length of stimulation study, 60 minutes of stimulation produced the greatest increase in tPA gene expression (17-fold compared with control). In measurements of bFGF, PDGF-B and TGF-1 expression, electrical stimulation produced the greatest effect on TGF-1 expression, where expression was enhanced up to 10 fold after 24 hours. BFGF and PDGF-B expression levels were similar to those seen in unstimulated controls.

### Example 5

ERP Promoters Linked to a Heterologous Gene

To test whether genetically engineered 293 cells or QBI-293 cells containing the ERP promoters would respond to electrical stimulation, the cells were transfected with a luciferase reporter gene attached to an electrically responsive promoter derived from atrial natriuretic factor. The transfected cells were subjected to an electric field for various time periods at 37° C. using the testing device previously described.

Cells were harvested and quantified by luciferase expression using a commercially available dual-luciferase promoter (DLR) assay kit (Promega Corporation) and a TD-20/20 luminometer (Turner designs). Differences in cell transfection efficiency between stimulation wells can be normalized to that produced from constitutive expression of pRLSV40.

FIG. 6 illustrates electrical stimulation enhanced the expression of luciferase in QBI-293A cells transfected with p638ANFluc. Cells were transfected with p638ANFluc as described herein. Twenty-four hours after transfection, cells were simulated for 24 hours under various conditions: (1) 10 Hz, 20 ms, 1 mA, 1.3 s polarity reversal; (2)10 Hz, 10 ms, 4 mA, 6.0 s polarity reversal; (3) 10 Hz, 20 ms, 1 mA, 6.0 s polarity reversal; (4) 5 Hz, 5 ms, 2 mA, AC coupled; (5) 10 Hz, 20 ms, 1 mA, AC coupled. After twenty-four hours of stimulation cells were harvested, and luciferase expression quantified.

FIG. 7 shows the time course of luciferase expression in p638ANFluc transfected QBI-293 cells after electrical stimulation. Cells were electrically stimulated at 10 Hz, 20 ms, 1 mA, 1.3 s polarity reversal. Electrical stimulation elicited a maximal 2.4 fold enhancement of luciferase expression after twenty-four hours, but enhanced expression was evident after 1 hour of stimulation.

FIG. 8 shows the time course for activation of luciferase in C₂C₁₂ cells after electrical stimulation. Cells transfected with p638ANFluc were electrically stimulated (10 Hz, 20 ms, 1 mA, 1.3 sec polarity reversal) for various time points up to twenty-four hours. C₂C₁₂ cells showed near maximal enhancement of luciferase expression at 20 minutes of stimulation.

### Example 6

Isolation and Culture of Satellite Cells

Masseter muscle samples were taken from anesthetized dogs under sterile conditions. Muscle samples were rinsed in 70% ethanol followed by three rinses in Hank's basal salt solution without calcium and magnesium, but containing 1% penicillin-streptomycin. Tissues were minced (˜1 mm³) before being incubated with 25 ml of enzyme solution (buffered medium 199; 1% collagenase; and 0.2% hyaluronidase filtered through a 0.2 μ filter and equilibrated with 95% O₂:5% CO₂) in a sterile 50 ml plastic centrifuge tube. After 15 minutes of incubation at 37° C. in a shaking water bath, the satellite cells are harvested by pouring the solution through layers of sterile gauze into a sterile container and pelleted by centrifugation. The remaining tissue was incubated in buffered medium 199 containing 1% protease at 37° C. for 15 minutes to complete the enzymatic release of satellite cells from muscle and processed. The packed cells are washed with medium 199 containing serum (10% fetal bovine serum) and 1% antibiotic antimycotic solution (Sigma Chemical Co., St Louis, Mo.) for 3 times by centrifugation (650×G for 10 minutes) and resuspended. Cell viability was checked by trypan blue exclusion and cell number determined by hemacytometry. Cells were diluted to 1 ×10⁶ cells with 8 ml of proliferation medium in a 25 cm² culture flask.

Cultured satellite cells were subcultured every 3 to 4 days to at low density for continued proliferation without differentiation. Recovered cells were rinsed with medium 199 before incubation with the same medium containing 1% protease at 37° C. for 10 minutes. The recovered cells were cultured with the procedure. To form multinucleated myotubes, the satellite cells were cultured with medium 199 containing 2% horse serum (Gibco, Grand Island, N.Y.) and 1% antibiotic antimytotic solution until myotube formation.

Normally, 4×10⁶ satellite cells with better than 90% viability were isolated from the muscle. The isolated cells were observed to have a doubling time of 20 to 22 hours and were able to go through atleat 20 cell cycles and still retain their proliferation and differentiation capabilities.

### Example 7

Labeling Cultured Satellite Cells

The mammalian reporter vector pCMVβ containing the lacZ gene, which encodes β-galactosidase, was originally purchased from Clontech Laboratory Inc. (Palo Alto, Calif.) and the lipofectamine reagent was obtained from Gibco BRL (Gaithersburg, Md.).

Transfection medium containing 50 μg of pCMVβ DNA and 220 μl (2 mg/ml) of lipofectamine in 10 ml of medium is incubated for 45 minutes and subsequently diluted to 50 ml with medium 199. To transfect cultured satellite cells (about 60% confluent) the cells are rinsed twice with medium 199 before overlaying with 3 ml of the DNA-liposome transfection medium. After 8 hours at 37° C. in a CO₂ incubator, 3 ml of 2× serum are added. Twenty-four hours after transfection, the transfection medium is replaced by growth medium. Forty-eight to 72 hours after transfection, X-gal histochemical staining is used to monitor the transfection. Greater than 90% of cells were found to produce β-galactosidase. From applicants' and others' studies, introduction of the lacZ gene does not interfere with the proliferation or differentiation of satellite cells.

### Example 8

Implantation of Labeled Cells into the Myocardium after Ischemic Injury

Under full anesthesia and sterile surgical conditions the heart is exposed through a midline stemotomy. The pericardium and the edges attached to the chest wall are opened to expose the left ventricle. After administration of heparin (100 U/kg) and lidocaine (2 mg/kg), the left anterior descending coronary artery (LAD) is temporarily occluded for 2 hours before reperfusion. The site of occlusion is just below the first branch of the LAD that is about two-thirds from the apex of the heart. This generally produces a reproducible myocardial infarction with low mortality (<5%). The ischemic myocardium can be identified by cyanosis and hypokinesis. The ischemic area is encircled with a 5-0 polypropylene suture for future identification.

After releasing the occluded LAD, cardiac function was stabilized before cell implantation. Five animals were randomly assigned to one of the following treatments: (1) injection of satellite cells into the myocardial infarction with a 25-gauge needle; (2) delivery of satellite cells into injured heart muscle using a Medtronic catheter; (3) injection of culture medium into infarcted myocardium

Neomyocardium Histology

Heart tissue was encased in 4% agarose and sectioned into 5 mm slices and reacted with X-gal. Sections were scanned and the fraction of normal, X-gal positive, and scar tissue was quantified. Histological and immunohistological evaluations were also performed.

### Example 9

Isolation and Culturing Skeletal Myoblast

The following solutions and materials were used in the isolation and culturing of skeletal myoblasts: 1) Isolation Medium: 80.6% M199 (Sigma, M-4530), 7.4% MEM (Sigma, M-4655), 10% Fetal Bovine Serum (Hyclone, Cat.#A-1115-L), 2× (2%) Penicillin/Streptomycin (Final Conc. 200,000 U/L Pen./20 mg/L Strep., Sigma, P-0781); 2) Myoblast Growth Medium: 81.6% M199 (Sigma, M-4530), 7.4% MEM (Sigma, M-4655), 10% Fetal Bovine Serum (Hyclone, Cat.#A-1115-L), 1× (1%) Penicillin/Streptomycin (Final Conc. 100,000 U/L Pen./10 mg/L Strep., Sigma, P-0781); 3) Wash Solution: M199, 2× Penicillin/Streptomycin; 4) Enzyme Solution: Prepare the enzyme solution, the same day it will be used, by adding 1.0 gm collagenase and 0.2 gm hyaluronidase to 100 ml of M199 (100 ml of enzyme/disbursing solution is enough to digest 40-50 gm of skeletal muscle). Filter sterilize the enzyme solution first through a 0.45 μm filter and then a 0.22 μm filter and keep at 4° C. until ready to use; 4) Disbursing Solution: Prepared the same day it will be used, by adding 1 gm of the protease (Protease, from Streptomyces griseus, (Sigma, P-8811). to 100 ml of M199. Filter sterilize through a 0.22 μm filter and keep at 4° C. until ready to use.

The following specialty reagents were obtained from the same vendor: Collagenase (Crude: Type IA, Sigma, C-2674); Hyaluronidase (Type I-S, Sigma, H-3506); Percoll (Sigma, P-4937); Trypsin Solution (Sigma, T-3924); BIOCOAT Laminin Cellware (25 cm² and 75 cm² flasks, Becton Dickinson, Cat. No(s). 40533, 40522), Trypsinization Solution: HBSS with 0.5 g/l trypsin (Sigma, T-3924); Hank's Balanced Salt Soution (HBSS), Ca²⁺ and Mg²⁺ free (Sigma, H-6648).

Isolation of Skeletal Myoblasts

Skeletal muscle biopsy, preferably from the belly of the muscle was placed into Isolation Medium in a sterile centrifuge tube or media bottle (approximately 30 to 50 ml of Isolation Medium were added to a sterile centrifuge tube containing approximately 10 grams of biopsy or less; If up to 25 grams of biopsy were used, 50 ml of Isolation Medium were added to a 125 ml sterile media bottle) and placed on ice (approximate 4° C.). To mince the tissue the tissue was removed and placed on a sterile petri dish and the connective tissue was trimmed away. The tissue was rinsed with sterile 70% EtOH for 30 seconds and then the EtOH was aspirated away from the tissue. The tissue was rinsed with 2× HBSS and finely minced with scissors and tweezers. The minced biopsy was transferred into 50 ml sterile centrifuge tubes. No more than 20 gm of tissue was added per tube. Approximately 25 ml of HBSS was added to each tube, mixed, and pelleted by centrifuging briefly at 2000 RPM in a Beckman Centrifuge, GS-6. The HBSS was decanted off and the tissue was again rinsed and centrifuged two more times. Enzyme Solution was added to the tubes (approximately 25 ml/15-20 gm original biopsy), and incubated in an incubator shaker for 20 minutes at 37° C., 300 RPM. The tissue was then centrifuged at 2000 RPM for 5 minutes and the supernatant was discarded. Disbursing Solution was added to the tubes (approximately 25 ml/15-20 grams original biopsy) and incubated in an incubator shaker for 15 minutes at 37° C. at 300 RPM. The sample was then centrifuged at 2000 RPM for 5 minutes and the supernatant was harvested and inactivated by by adding Fetal Bovine Serum to a final concentration of 10% (10% Fetal Bovine Serum (Hyclone, Cat.#A1115-L) and stored at 4° C. Disbursing Solution was added to the tubes for a second enzymatic digestion, incubation, and isolation. The cell suspension slurries from the disbursing digestion steps were centrifuged at 2400 RPM for 10 minutes. The cell pellet was resuspended in a minimal volume of Wash Solution and the pellets combined into a 50 ml centrifuge tube and the final volume adjusted to 40 ml with Wash Solution. The cells were again centrifuged at 2400 RPM for 10 minutes to isolate the cells. The cells were washed two more times with Wash Solution and finally resuspended in 2-4 ml of MEM depending whether starting with more or less than 25 gm of tissue. Approximately 2 ml of cells were layered onto 10 ml of 20% Percoll/MEM over 5 ml of 60% Percoll/MEM. Cells were centrifuged at 11947 RPM (15000×g) for 5 minutes at 8° C. and the band of cells that develops between the 20% and 60% Percoll layers was isolated. This band contains the myoblast cells. The collected ban of cells was diluted with 5 volumes of growth medium and again centrifuge at 3000 RPM for 10 minutes. The supernatant was removed and the cells were resuspended in growth medium, counted, and plated on BIOCOAT Laminin coated T-flasks at approximately 1×104 cells/cm2 (the first plating should be done on a laminin coated surface to aid in cell attachment). Cells were cultured to 60% -80% confluence and then passed before the cells become terminally differentiated.

Culturing Skeletal Myoblast Cells

Growth Medium Formulation consists of 81.6% M199 (Sigma, M-4530), 7.4% MEM (Sigma, M-4655), 10% Fetal Bovine Serum (Hyclone, Cat.#A-1115-L), and 1× (1%) penicillin/streptomycin (Final Conc. 100,000 U/L Pen./10 mg/L Strep., Sigma, P-0781). Generally cells are passed at a seeding density of 1×104 cells/cm2. Typically this will yield an 80% confluent monolayer in approximately 96 hours. Similarly, cells can be split at ratios of 1:4-1:6, which will yield a confluent monolayer within 96 hours. To effectively pass the cells, the cells are not allowed to become confluent. Cell to cell contact will cause the cells to differentiate into myotubes.

In order to pass the cells the culture medium was first removed from T-flask. The appropriate amount of Hank's Balanced Salt Solution (HBSS) was added back to the flask and incubated for approximately 5 minutes at room temperature. The HBSS was removed and replace with the Trypsin solution and incubated for a maximum of 5 minutes at 37° C. in a 5% CO2 incubator. Gentile agitation helps remove cells. The flask was diluted with at least an equal volume of growth medium to neutralize the trypsin. A sample was removed and counted and then the cells were centrifuged at 800-1000 RPM for 10 minutes. The cells were recounted and resuspended in cell culture medium and seeded into appropriate flasks. To maintain a healthy culture, media was changed every 2-3 days.

### Example 10

Electrical Stimulation of Transplanted Cells In Myoinfarcted Tissue

Myocardial infarction was induced in fifteen canines by temporary coronary artery occlusion (LAD) followed by reperfusion. Following the infarction/reperfusion, animals in the control group received injections of culture medium, animals in the test group 1 received 5×10⁷ skeletal myoblast cells directly injected with a syringe and the animals in test group 2 received 5×10⁷ skeletal myoblast cells delivered by a prototype Medtronic catheter. Six weeks after the initial surgery, animals were instrumented with sensors to measure their cardiac function and were sacrificed. Eight of the animals were additionally electrically stimulated during the cardiovascular functional studies.

Histological sections of the infarct regions were stained with Masson's Trichrome. Transplanted cells were visualized by X-gal histochemical staining. Results showed that animals in both test group 1 and test group 2 developed healthy looking muscle tissue at the implant site. Furthermore, there was no discernible difference in the new muscle structure for cells injected by needle versus cells injected by a Medtronic catheter. In the control animals, the infarct region had abundant connective tissue formed by fibrin and collagen, without evidence of cardiocytes.

Cardiac function was evaluated using pressure—segment length loops. The infarct areas of the hearts receiving cell replacement therapy maintained an elastic structure while the infarcts in the control hearts gained more plastic properties. While electrical stimulation had no significant benefit for the three control animals, three of the five animals receiving cell replacement therapy showed at least a 40% increase in cardiac function with the application of the electrical stimulation.

Histopathological Methods and Results

In order to assure that the transplanted skeletal cells were present at the end of the two week period, preserved tissue sections were analysed with immuno-histochemistry using an anti-myosin antibody (Monoclonal Anti-Skeletal Myosin (Fast), clone MY-32, Sigma, Cat.No. M-4276.). Positive (green) staining at two different regions of the ablated site indicated the presence of the injected skeletal muscle cells in the ablated region of myocardium, two weeks after their introduction. This immuno-staining study provided definitive evidence for the presence of skeletal muscle cells in the myocardium.

The complete disclosures of the patents, patent applications, and publications listed herein are incorporated by reference, as if each were individually incorporated by reference. The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

### References

U.S. Pat. No. 4,683,195, Mullis, K. B., issued Jul. 28, (1987).

WO 00/18903, Marban, E., et al., Inducible genetic suppression of cellular excitability (2000).

Annex, B. H., et al., Induction and maintenance of increased VEGF protein by chromic motor nerve stimulation in skeletal muscle, Am. J. Physiol. 274(43), H860-H867 (1998).

Bessereau, J-L et al., In vivo and in vitro analysis of electrical activity-dependent expression of muscle acetylcholine receptor genes using adenovirus, Proc. Natl. Acad. Sci. USA, 91, 1304-1308 (1994).

Calvo, S., et al., Transcriptional control of muscle plasticity: differential regulation of tropinin I genes by electrical activity, Dev. Genet, 19(2), 169-181 (1996).

Cresci, S., et al., Activation of a novel metabolic gene regulatory pathway by chromic stimulation of skeletal muscle, Am. J. Physiol. 270, C1413-C1420 (1996).

Fitzsimmons, R. J., et al., Low-amplitude, low-frequency electric field-stimulated bone cell proliferation may in part be mediated by increased IGF-II release, J. of Cellular Physiol. 150, 84-89 (1992).

Franz, W-M., et al., Heart-specific targeting of firefly luciferase by the myosin light chain-2 promoter and developmental regulation in transgenic mice. Circ. Res. October;73(4):629-38 (1993).

Fukamizu, A., et al., Tissue-specific trans-activation of renin gene by targeted expression of adenovirus E1A in transgenic mice. Biochem Biophys Res Commun. Feb 28, 1994;199(1):183-90 (1994).

Graef, I. A., et al., L-type calcium channels and GSK-3 regulate the activity of NF-Atc4 in hippocampal neurons, Nature (London) 401(6754), 703-708 (1999).

Gilmour, B. P., Electrical activity suppresses nicotinic acetylcholine receptor γ subunit promoter activity, Developmental Biology 168, 416-428.

Hang, J., et al., (1995), VEGF gene expression is upregulated in electrically stimulated rat skeletal muscle, Am. J. Physiol. 269, H1827-H1831 (1995).

Gulick, et al., Isolation and characterization of the mouse cardiac myosin heavy chain genes, J Biol Chem. 266(14):9180-5 (1991).

Gustafson, T. A., et al., Thyroid hormone regulates expression of a transfected alpha-myosin heavy-chain fusion gene in fetal heart cells. Proc Natl Acad Sci U S A. May;84(10):3122-6 (1987).

Hang, J., et al., VEGF gene expression is upregulated in electrically stimulated rat sketeletal muscle, Am. J. Physiol. 269(38), H1827-1831 (1995).

Inoue, A., et al., The human preproendothelin-1 gene. Complete nucleotide sequence and regulation of expression. J. Biol Chem. September 5;264(25): 14954-14959 (1989).

Jahrondi, N., et al. Mol. Cell. Boil. 14:999-1008 (1994).

Kanno, S., et al., Establishment of a simple and practial procedure applicable to therapeutic angiogenesis, Circulation, 99, 2682-2687 (1999).

Kaye, D. M., et al., Frequency-dependent activation of a constitutive nitric oxide synthase and regulation of contractile function in adult rat ventricular myocytes, Circulation Research 78(2), 217-224 (1996).

Knowlton, K. U., et al., Co-regulation of the atrial natriuretic factor and cardiac myosin light chain-2 gnes during α-adrenergic stimuation of neonatal rat ventricular cells, J. Biol. Chem., 266, 7759-7768 (1991).

Lin, H., et al., Specific region of the c-myc promoter is responsive to electric and magnetic fields, J. of Cellular Biochemistry 54, 281-288 (1994).

Luke, M. C., et al., Human androgen receptor binding to the androgen response element of prostate specific antigen. J. Androl. January-February;15(1):41-51 (1994).

Macri, P., et al., Hum. Gene Ther. 5:175 (1994).

Mahdavi, V., et al., Cardiac alpha- and beta-myosin heavy chain genes are organized in tandem, Proc Natl Acad Sci U S A. 81(9):2626-30 (1984).

Mao, Z., et al., Neuronal activity-dependent cell survival mediated by transcription factor MEF2, Science 286, 785-790 (1999).

McDonough, P. M., et al., (1992), Induction of atrial natriuretic factor and myosin light chain-2 gene expression in cultured ventricular myocytes by electrical stimulation of contraction, J. Biol. Chem. 267, 11665-11666 (1992).

McDonough, P. M., et al, Involvement of cytoplasm calcium and protein kinases in the regulation of atrial natriuretic factor secretion by contraction rate and endothelin, J. Biol. Chem. 269(13), 9466-9472 (1994).

McDonough, P. M., et al., Collaborative roles for c-Jun N-terminal kinase, c-Jun serum response factor, and Sp1 in calcium-regulated myocardial gene expression, J. Biol. Chem., 272(38) 24046-24053 (1997).

Minty, A., et al., Upstream regions of the human cardiac actin gene that modulate its transcription in muscle cells: presence of an evolutionarily conserved repeated motif, Mol Cell Biol. June;6(6):2125-36 (1986).

Molkentin, J. D., et al., Transcription factor GATA-4 regulates cardiac muscle-specific expression of the alpha-myosin heavy-chain gene. Mol Cell Biol July; 14(7): 4947-4957 (1994).

Murry, C., et al., (1996), Skeletal myoblast transplantation for repair of myocardial necrosis, J. Clin. Invest 98(11), 2512-2523 (1984).

Murtha, P., et al., Androgen induction of a human prostate-specific kallikrein, hKLK2: characterization of an androgen response element in the 5′ promoter region of the gene. Biochemistry Jun 29, 1993;32(25):6459-64 (1993).

Nattel, S., Atrial electrophysiological remodeling caused by rapid atrial activation:underlying mechanisms and clinical relevance to atrial fibrillation, Cardiovascular Research 42, 298-308 (1999).

Schiaffino, S., et al., Neural regulation of myosin gene expression in regenerating skeletal muscle, Acta Physiol. Scand. 163, S11-S15 (1998).

Sasaoka, T., et al., Analysis of the human tyrosine hydroxylase promoter-chloramphenicol acetyltransferase chimeric gene expression in transgenic mice. Brain Res Mol Brain Res Dec;16, 1992(3-4):274-86 (1992).

Skarli, M., et al (1995), Muscle specific promoters in gene therapy of hemophilia and muscular dystrophy-recombinant somatomedic-C expression in muscle fiber by tissue specific gene expression; potential gene therapy, J. Cell. Biochem. Suppl21A, 383 (1995).

Sprenkle, A. B., et al., (1995), Involvement of multiple cis elements in basal- and α-adrenergic agonist-inducible atrial natriuretic factor transcription, roles for serum response elements and SP-1-like element, Circ.Res. 77, 1060-1069 (1995).

Tabuchi, A., et al., Differential activation of brain-derived neurotrophic factor gene promoters I and III by Ca2+ signals evoked via L-type voltage-dependent and N-methyl-D-aspartate receptor Ca2+ channels, J. of Biol. Chem. 275(23), 17269-17275 (2000).

Thelen, M H. M., et al., (1997) Electical stimulation of C2C12 motubes induces contractions and represses thyroid-hormone-dependent transcription of the fast-type sarcoplasric-reticiulum Ca2+-ATPase gene., Biochem J. 321,845-848 (1997).

Ventura, C., et al., Elf-pulsed magnetic fields modulate opiod peptide gene expression in myocardial cells, Cardiovascular Research 45, 1054-1064 (2000).

Vilbert, M., et al., Eur. J. Biochem. 181:33 (1989).

Xia, Y., et al., Changes in expression of heart carnitine palmitoyltransferase I isoforms with electrical stimulation of culture rat neonatal cardiac myocytes., 271(20), 12082-12087 (1996).

Xia, Y., et. al., Electrical stimulation of neonatal cardiomyocytes results in the sequential activation of nuclear genes governing mitochondrial proliferation and differentiation, Proc. Natl. Acad. Sci. 94, 11399-11404 (1997).

Xia, Y., et al., Activation of the cytochrome c gene by electrical stimulation in neonatal rat cardiac myocytes, J. Biol. Chem. 273(20), 12593-12598 (1998).

Xia, Y., et al., Electrical stimulation of neonatal cardiac myocytes activates the NFAT3 and GATA4 pathways and up-regulates the adenylosuccinate synthetase 1 gene, J. Biol. Chem., 275(3), 1855-1863 (2000).

Yanagida, Y., et al., Electrically stimulated induction of hsp70gene expression in mouse astroglia and fibroblast cells, 79,53-61 (2000).

Zhang, H., et al., Dual tandem promoter elements containing CCAC-like motifs from the tetrodoxtoxin-resistant voltage-sensitive Na+ channel (rKkM2) gene can independently drive muscle-specific transcription in L6 cells (1999).

What is claimed is:

1. A therapeutic delivery system comprising an electrical pulse generator operably coupled with genetically engineered cells in a mammalian tissue, wherein said genetically engineered cells further comprise a target gene operably coupled to an electrically responsive promoter.

2. A therapeutic delivery system of claim 1 wherein the electrical pulse generator provides a subthreshold stimulation.

3. A therapeutic delivery system of claim 1 wherein the electrical pulse generator provides a threshold stimulation.

4. A therapeutic delivery system of claim 1 wherein the electrical pulse generator provides stimulation to the tissue from attached electrodes.

5. A therapeutic delivery system of claim 1 wherein the electrical pulse generator provides stimulation to the tissue without attached electrodes using Eddy currents induced by time varying magnetic fields.

6. A therapeutic delivery system of claim 1 wherein the electrical pulse generator provides stimulation to the tissue without attached electrodes using displacement currents induced by time varying electrical fields applied externally.

7. A therapeutic delivery system of claim 1 wherein the electrical pulse generator is a pacemaker.

8. A therapeutic delivery system of claim 1 wherein the electrical pulse generator is implanted.

9. A therapeutic delivery system of claim 1 wherein the electrical pulse generator is external

10. A therapeutic delivery system of claim 1 wherein the electrical pulse generator is externally controlled.

11. A therapeutic delivery system of claim 1 wherein the electrical response promoter contains an electrically responsive enhancer element that is heterologous to the coding sequence.

12. A therapeutic delivery system of claim 1 wherein the electrical response promoter contains an electrically responsive enhancer element heterologous to the promoter sequence.

13. A therapeutic delivery system of claim 1 wherein the electrically responsive promoter is responsive to subthreshold stimulation.

14. A therapeutic delivery system of claim 1 wherein the electrically responsive is responsive to threshold stimulation.

15. A therapeutic delivery system of claim 1 wherein the electrically responsive promoter contains an electrically responsive enhancer element selected from the ANF 5′ non-coding region.

16. A therapeutic delivery system of claim 1 wherein the electrically responsive promoter comprises an ERE operably linked to a tissue specific promoter.

17. A therapeutic delivery system of claim 1, wherein said promoter is a cardiac-specific promoter.

18. A therapeutic delivery system of claim 17, wherein said promoter is selected from the group consisting of the ANF promoter, alpha-MHC.sub.5.5 promoter, alpha-MHC.sub.87 promoter, and human cardiac actin promoter.

19. A therapeutic delivery system of claim 1, wherein said promoter is a kidney specific promoter.

20. 6. A therapeutic delivery system of claim 1, wherein said promoter is a brain specific promoter.

21. A therapeutic delivery system of claim 1, wherein said promoter is selected from the group consisting of aldolase C promoter, and tyrosine hydroxylase promoter.

22. A therapeutic delivery system of claim 1, wherein said promoter is a vascular endothelium specific promoter.

23. A therapeutic delivery system of claim 1, wherein said electrical response promoter, or fragment thereof, is selected from the group consisting of ANF, VEGF, acetylcholine receptor, troponin, NOS3, cytochrome c, COX, CPT-1, hsp70, and skm2.

24. A therapeutic delivery system of claim 1 wherein the genetically engineered cells are mammalian cells.

25. A therapeutic delivery system of claim 1 wherein the genetically engineered cells are selected from the group of C2C12.

26. A therapeutic delivery system of claim 1 wherein said coding sequence is selected from the group consisting of tissue plasminogen activator (tPA), nitric oxide synthase (NOS), Bcl-2, superoxide dismutase (SOD), and catalase.

27. An expression vector, comprising an electrical response enhancer element, a tissue specific promoter heterologous to the element, and a coding sequence, wherein said promoter is operably linked to said coding sequence and said element is effective to cause expression of said coding sequence.

28. An expression vector of claim 27, wherein said expression vector is a plasmid.

29. An expression vector of claim 27, wherein said expression vector is an adenovirus vector.

30. An expression vector of claim 27, wherein said expression vector is a retrovirus vector.

31. An expression vector of claim 27, wherein said coding sequence is a viral thymidine kinase coding sequence.

32. An expression vector of claim 31, wherein said viral thymidine kinase coding sequence encodes herpes simplex viral thymidine kinase.

33. An expression vector of claim 27, wherein said coding sequence encodes luciferase.

34. An apparatus for testing cells comprising an upper plate electrode, a lower plate electrode, and a porous membrane which is positioned between said upper and lower plate electrodes during operation.

35. An apparatus of claim 34 wherein the upper plate electrode is the same size as the lower plate electrode.

36. An apparatus of claim 34 wherein the lower plate electrode forms a receiving means for the porous membrane.

37. An apparatus of claim 34 wherein the porous membrane supports cells between said upper and lower plate electrodes.

38. An apparatus of claim 34 which is operably coupled to a pulse generator.

39. A method of treating a patient comprising providing the patient with a electrical pulse generator operably coupled with genetically engineered cells in a patient tissue, wherein said genetically engineered cells further comprise a target gene operably coupled to an electrically responsive promoter.

40. A method providing a patient with a electrical pulse generator operably coupled with genetically engineered cells in a patient tissue, wherein said genetically engineered cells further comprise a target gene operably coupled to an electrically.

41. A genetically engineered cell of claims 1, 39 or 40 wherein genetically engineered cells a transplanted into the patient tissue.

42. A method of either claims 1, 39 or 40 wherein genetically engineered cells are obtained by transfecting the cells of the patient tissue.

43. A method of either claims 1, 39 or 40 wherein the transfected tissues are independently selected from, epithelial tissue, endothelial tissue, or mesodermal tissue.

44. A genetically engineered cell of claims 1, 39, or 40 independently selected from the group consisting skeletal muscle cells, heart muscle cells, smooth muscle cells, pluripotent stem cells, mesodermal stem cells, myoblast, fibroblasts, cardiomyocytes, cholinergic neurons, andrenergic neurons, and peptidergic neurons, gial cells, astrocytes, oligodendrytes, schwann cells. vascular endothelial cells, synovial cells, acinar cells, hepatocytes, chondrocytes, osteoblasts, osteoprogenitor cells, nucleous pulposus cells, and cells of the intervertebral disk.
